(19) United States
(12) Patent Application Publication
(10) Pub. No.: US 2019/0000795 A1
(43) Pub. Date: Jan. 3, 2019

(54) Methods of using cannabinoids and/or molecular similars for modulating, waking-up and/or disabling cellular functions involved in causing disease -- reinvigorating metabolism with anandamide, 2-arachidonoylglycerol and similarly acting compounds

(71) Applicant: Richard Postrel, Miami Beach, FL (US)

(72) Inventor: Richard Postrel, Miami Beach, FL (US)

(21) Appl. No.: 15/952,237

(22) Filed: Apr. 13, 2018

Related U.S. Application Data
(60) Provisional application No. 62/526,569, filed on Jun. 29, 2017.

(51) Int. Cl. A61K 31/352; A61K 31/05; A61K 31/192; A61K 31/01; A61K 31/59; A61K 31/519; A61K 31/522; A61K 31/07; A61K 31/155; A61K 31/047; A61K 31/5377

(52) U.S. Cl. CPC A61K 31/352; A61K 31/05; A61K 31/192; A61K 31/01; A61K 31/59; A61K 31/5377; A61K 31/522; A61K 31/07; A61K 31/155; A61K 31/047; A61K 31/519

(57) ABSTRACT

Methods using cannabinoids and/or molecular similars for modulating, waking-up and/or disabling cellular functions involved in causing disease are provided for reinvigorating metabolism, including human metabolism. As humans age stresses they encounter cause their cells to respond opportunistically to counter each stress. Each of these responses involves at least minor shifts or compromises in metabolism. As these metabolic compromises multiply, metabolism continues to deviate from its optimum. Several lipophilic compounds are active in membranes of cells, including the plasma membrane, nuclear membrane, endoplasmic reticulum membrane and mitochondrial membrane. These lipophilic compounds, act with cannabinoid receptors to affect virtually all cell functions. By choosing specific endocannabinoids and/or analogues and targeting them at opportunistically deteriorated portions of metabolism through one of several specific endocannabinoid receptor proteins in a stepwise manner metabolism can be reinvigorated towards its optimal status.

Life is a complicated interaction of physics (movement—including temperature and sound, light, mass attraction, electricity, magnetism), chemistry (interaction of electrons amongst atoms putting together molecules and these larger structures and their functionalities), and biology (melding of chemistry and physics to produce organisms capable of making like organisms [self-replication]). Biology is the science of life, a process embodying sentience, sharing of experiences, remote sensing and transfer of experiences and, as evidenced in humans, ability to understand and improve life itself.

The present invention features compositions and methods that balance the complex interactions between and within cells to reinvigorate metabolism in receptive cells and to improve health and life functions of the organism as its entirety. Especially featured are several lipophilic compounds, compounds that easily interact with and often easily cross plasma and intracellular membranes to interact with a set of receptors that affect virtually all cell systems either directly or indirectly. Application of one or more of these compositions to ameliorate stress conditions or disease benefits the receptive cells and feedbacks throughout intercellular communication systems to benefit the entire organism.

Even in single celled organisms, the molecules in the cells apply physics (movement) and chemistry (making and breaking chemical compounds) to deviate from and to maintain a status of equilibrium. They concentrate chemical elements and build them into molecules to collect, concentrate and build more molecules to an extent sufficient to produce daughter cells.

Multicellular organisms sport many different types of cells with specialized functions. As organisms grew, laws of physics prevented adequate delivery through simple diffusion of the chemical substances required for further growth. The organism assigned different cells expedite the cooperation between cells organisms developed chemical messengers allowing one part of a cell to control activities in another cell part and crucially to allow cells in one part of the organism to signal needs and supply support to distant cells.

A classical illustration of this is called “The endocrine system”, specialized tissues or glands that sense their surroundings and dispatch hormones to the rest of the organism. Classical endocrine glands have specialized cells that produce hormones, chemicals proprietary to that gland that are secreted into circulation to inform and instruct distant cells. These glands include the hypothalamus, pituitary, thyroid, parathyroid, adrenal, thymus, pineal body, ovaries, testes and pancreas.

However, cells not specialized for communication also benefit from communicating with other cells, both neighboring and more distant. For example, simple metabolites such as CO₂ and H⁺ act as messengers to surrounding cells (and when carried in circulation to central systemic areas like the brain) can affect the whole organism.

Communication between cells is broken down into four categories. The brain is the central communications hub for the neural system which combines electromagnetism and chemical diffusion to rapidly transmit signals from one location to another specific location. The endocrine system is slower, using chemicals carried through the circulation. The endocrine system glands make and release chemicals in accordance with signals they receive (chemical or physical) to other tissues connected through the circulatory system. These signals may be specific to only a few cells or may be acted upon by many cells. Cells having a chemical that binds to the chemical released by the endocrine gland will respond. Cells without the “receptor” simply do not see the hormone and thus ignore it.

Paracrine signaling works by diffusion and involves chemicals released from on cell moving by diffusion to neighboring cells. Diffusion is a slow process dependent on size of the molecule. And when cells are in contact with one another they can communicate through these contacts using signaling, sometimes even forming tubes connecting their plasma membranes. Specialized proteins called “notch” receptors when they contact and bind to a specific ligand on a neighboring cell are chemically altered to initiate a signaling cascade of chemical reactions within the “notched” cell.

And some cells secrete compounds that activate the secreting cell. This is called autocrine signaling. Autocrine signaling that involves an interstitial sided receptor can be modulated or blocked by other signaling systems. Thus, a cell's responses that include secreting a compound that can act as a ligand for a membrane receptor on that that secreting cell are fine-tuned by actions of other cells or by other chemicals present in the organism at that time.

Some compounds have multiple functions, sometimes being carried through the circulation, but also acting more locally and even for compounds such as NO and reactive oxygen species (ROS) can work through multiple systems, from intracellular communication to paracrine communication and in several cases through the circulation.

Cannabinoids are lipid-based compounds that can be divided into endogenous cannabinoids (endocannabinoids), which are generated naturally inside the body, from exogenous cannabinoids, which are introduced into the body as cannabis or a related synthetic compound. The endogenous cannabinoid ligands are unsaturated fatty-acid ethanolamides, glycerols or glycerol ethers. The endocannabinoids are vital and ancient signaling molecules with evidence in yeast, helminthes, and most advanced organisms, including mammals.

Cannabinoid receptors are located throughout the cells of our bodies. These receptors are features of or endocannabinoid endocrine system which is involved in a variety of physiological processes including, but not limited to: appetite, pain-sensation, activity mood, and memory. The pervasive distribution of cannabinoid receptors allows their manipulation to have profound effects depending on the cannabinoid utilized and the temporal and physical distribution of the relevant receptor. The cannabinoid system has autocrine, juxtacrine (through direct transfer by membrane lipids in contact), paracrine and given their heavy involvement in the nervous system, neural communication, though they appear to act as modulators and intermediaries in this communication system.

Cannabinoids have benefits beyond their communicating functions. U.S. Pat. No. 6,630,507, Cannabinoids as antioxidants and neuroprotectants, assigned to US Dept of Health and Human Services, proposes cannabinoids as a template for a set of antioxidant drugs. The disclosed invention “includes methods of preventing or treating diseases caused by oxidative stress, such as neuronal hypoxia, by administering a prophylactic or therapeutically effective amount of a cannabinoid to a subject who has a disease caused by oxidative stress.”

Classic cannabinoid receptors, CB₁ and CB₂ are members of a class of cell membrane receptor G protein-coupled receptor superfamily. G protein-coupled receptors typically comprise seven transmembrane spanning domains, as do both CB₁ and CB₂. These cannabinoid receptors are activated by three major groups of ligands: endocannabinoids, produced by the mammillary body; plant cannabinoids (such as tetrahydrocannabinol (THC), tetrahydro-cannabinolic acid (THCA) and cannabidiol (CBD), products of the cannabis plant); and synthetic cannabinoids (such as HU-210). Several less well characterized phytocompounds are known. These include: cannabidivarin (CBDV), cannabidiol acid (CBDA), cannabidivarin acid (CBDVA), cannabichromene (CBC), cannabigerol (CBG), cannabigerol acid (CBGA), cannabigerovarin (BGV), cannabinol (CBN), cannabinovarin (CBNV), tetrahydrocannabivarin (THCV), tetrahydrocannabivarin acid (THCVA), delta-8 tetrahydrocannabinol (Δ⁸-THC), delta-9 tetrahydrocannabinol (Δ⁹-THC) (aka THC). CB₂ recognizes the same structural groups of cannabinoid agonists as CB₁. N-arachidonoyl-ethanolamine (AEA) has low CB₂ affinity, but 2AG binds well to both CB₁ and CB₂.

The cannabinoid receptors are participants in the endocannabinoid signaling system that includes enzymes that synthesize and degrade endocannabinoids. While the lipid ligands appear to freely traverse the plasma and intracellular (organelle based) lipid membranes, there may be relevant transport molecules with variable affinities for the endogenous ligands or synthetic analogues. Flexibility allows proper orientation in the membrane with the head peeking out at the level of phospholipids in the membrane and the tail meandering into the center. The receptor and degradative enzymes are all membranous and thus optimally situated for interaction with the cannabinoids.

When considering analogues or synthetics to use as mimetics or partial mimetics of agonists for these receptors the skilled artisan would take into account binding characteristics of the receptors. CB₁ binding requires at least a pentyl length alkyl tail. CB₂ readily binds shorter carbon chains, as short as dimethylethyl and dimethylpropyl. Thus, a CB₁ analogue based on a pentyl (5 carbon), hexyl (6 carbon), heptyl (7 carbon), etc., including carbon chains of 8, 9, 10, 11, 12, 13, 14, 15, 16, 17, 18, 19, 20, 21, 22, 23, 24, 25, etc. in length can be used. The number of carbons may actually be higher than the chain length due to branching or inclusion of ringed structures including aromatic and/or N or S containing chains or rings. CB₂ analogues would start with a lower number but would include ethyl (2 carbons), propyl (3 carbons), butyl (4 carbons and the longer chains as above listed for CB₁. Chain flexibility appears as a positive factor for improved ligand binding. Delivery of cannabinoids to cells is facilitated when the lipophilic compounds are compartmentalized into a membranous-like structure, especially a structure that can incorporate into a biomembrane which it contacts. Such combination can be facilitated and targeted at specific membranes using a principle akin to the endocrine system, building into the compartmentalizing membrane ligands appropriate for receptors on the targeted cell. These targeting ligands are preferably independent molecules of the transported cannabinoids allowing the recognition proteins to be independently changed to provide any selected endocannabinoid or analogue access to the receptive cell for incorporation into its membranes and contact with its cannabinoid receptors.

Synthetics may be modified with carriers, including labile or cleavable carriers. Increasing hydrophilicity of the ethanolamine portion of AEA or of the less lipophilic and therefore cytoplasmic or organelle matrical exposed region of analogously structured cannabinoids can help highlight their orientation and increase aqueous based interactions with aqueous faced receptors. Halogenation, oxidation, sulfur or sulfonation, additional nitrogen groups including those that may be cyclized can be used for this effect. Although the conjugated bonding provides stiffness to help orient the ligand in the membrane the length of the carbon tail and its unsaturated content can be programmed to target specific membranes, for example, mitochondrial membranes have higher protein content and greater fluidity of their lipids.

All known endocannabinoids and popular plant cannabinoids are lipophilic and therefore are often found associated with membranes. In blood, endogenous and external (phyto-) cannabinoids are associated with albumin or on lipoproteins. Synthetic cannabinoids may associate with proteins, lipoproteins, glycoproteins, fats and/or hydrating water molecule depending on solubility characteristics.

Therapeutic uses of exogenous cannabinoids have been traced back at least 4,000 years to ancient dynasties of China. Cannabinoid compositions have been applied for managing various illnesses including lack of appetite, emesis, cramps, menstrual pain, spasticity and rheumatism. This extensive medicinal application of cannabinoid substances has prompted the development of several modern pharmaceutical drugs. For example, SATIVEX™ (a combination of the natural substances THC and CBD), MARINOL™ (generic name dronabinol), CESAMET (generic name nabilone™), two synthetic cannabinoids based on THC, are prescribed anti-emetic and for enhancement of appetite, mainly in AIDS patients. In addition to their clinical use as an antiemetic, potential therapeutic uses of nonselective cannabinoid receptor agonists include the management of multiple sclerosis, spinal cord injury, pain, inflammatory disorders, glaucoma, bronchial asthma, vasodilatation that accompanies advanced cirrhosis, and cancer; DEXANABINOL™ (a synthetic non-psychotropic cannabinoid that blocks NMDA receptors and COX-2 cytokines and chemokines) proposed for memory restoration after traumatic brain injury; CT-3 (generic name ajulemic acid), a potent synthetic analog of the THC metabolite, THC-11-oic acid proposed for neuropathic pain and anti-inflammatory for arthritis; CANNABINOR™, failed Phase II(a) efficacy trial for reduced pain; HU-308, small molecule that specifically binds CB₂, proposed to treat high blood pressure and inflammation; HU-331, binds CB₁, CB₂, and non-specific receptors, proposed for a variety of therapies, including, memory, weight loss, appetite, analgesia, tumor surveillance, and inflammation; RIMONABANT™ (in Europe), (Acomplia™ in US) small molecule pharmaceutical that binds CB₁ and some CB₂, effective appetite suppressor, proposed as a smoking cessation aid, withdrawn from market due to psychiatric side effects; and TARANABANT™ (inverse agonist of brain CB₁), an appetite suppressant, but also found to have side effects of increased anxiety and depression, withdrawn from development; are or have been at various stages of development in the pharmaceutical pipeline.

When metabolism is functioning optimally, the cell delivers appropriate substrate, the substrate is processed and the products are delivered to the next step in that pathway. Optimal function requires proper interactions between scores of carrier molecules, activating enzymes, activated enzymes, tagging molecules, catabolic and anabolic enzymes, etc. The original ligand and then its metabolites must be in the proper position with all the reaction components properly positioned for optimal metabolism to proceed. When a reaction proceeds improperly a wrong metabolite may be presented thereby arresting the entire cascade because when an atypical result occurs, it may be impossible for the enzyme and atypical ligand to bind and then next reaction to occur.

This atypical product may be secreted from the cell, may be used in a different pathway, may bind or otherwise interfere with another molecule in the cell, may be degraded by scavenging actions within the cell, or may just float around getting in the way. Usually the cell has a means of correcting or discarding metabolic errors. But often when an unexpected substrate (perhaps a drug or toxin or just an unaccustomed food) or an abnormal amount of substrate presents, the cell will switch its biochemical machinery in response to the stress, perhaps activating a kinase, inducing transcription of an enzyme or receptor, tagging an enzyme for recycling, or epigenetically altering the activity of genetic material. Sometimes these changes are not easily reversible but managed in the cell. Sometimes these may lead to cell death through initiation of apoptosis. Generally, the adaptations trigger changes in related pathways which may produce a small or large imbalance of the cell's original metabolic status.

Small corrective adaptations especially when applied early before inordinate volumes of baggage have accrued can, given time and redirection of opportunistic metabolic actions, restore life and vitality to the organism and its cells. More vigorous metabolic steering may be warranted in situations where opportunistic behaviors have more seriously compromised beneficial metabolism.

Many diseases result not from one single metabolic mistake but evolve from a compounding of several events that eventually manifest as a disease state. For example, deceased vigor with age, Alzheimer's Disease (AD), cancers, several auto-immune diseases, and many progressive diseases involve multiple mutational and/or compensatory events for the cell's survival. From the cell's perspective, one metabolic change will affect all downstream pathways, some of which will involve feedback loops to one or more upstream paths. The initial event and compensatory responses may tilt the selective pressures such that events we might normally count as mistakes might in fact, from the cells position, merely be the most opportunistic response to improve continuity of that cell and its lineage. A short term advantage to a cell may often prove to be deleterious to the cell or organism in the long run.

Modifying the selective pressures to disfavor these compensatory secondary or tertiary modifications that are at that moment advantageous to the modified cell, but not to the organism's long term well-being, can prevent progression to or progression along a disease state. Recognizing the early “errors” and engaging the cell's or organism's compensatory mechanisms is a preferred and natural path for preventing or eliminating metabolic or proliferative disease or heightened risk of disease. Endocannabinoids or analogues of endocannabinoids can be used as corrective intervention to rebalance or redirect cells' diverted metabolisms.

Since metabolism is complex and comprises many different metabolic pathways that might, in response to momentary stress circumstance, maladapt and thereby lead to cell-survival-enhancing, but organism-degrading, opportunistic compensatory maladaptation, a large number of outside interventions are available as tools to rebalance the cells towards more long-term organism and less immediate cellular benefit. Many of the second, third, fourth, . . . , reactions will to some extent lessen the impact of the first maladaptation, for example, by providing less substrate (e.g., LeChatelier's feedback) when a the maladapted path less vigorously consumes a substrate; by activating a parallel, crossing or serial path when a product becomes in excess or an intermediate product is released; or up-regulating or down-regulating through another process, or e.g., through a more complex process perhaps involving stabilizing or catabolizing a protein, altering RNA metabolism, and/or activating or deactivating transcription factor pathways. Metabolism can be improved using signaling pathways such as supplements that agonize or antagonize the targeted pathways. Supplements may comprise a compound directly active at its target to elicit a response or may act indirectly, e.g., by inducing synthesis and/or secretion of a messenger substance to encourage metabolic refocusing. One example of a class of metabolic modifiers that may be manipulated directly or indirectly is those communicating with cannabinoid receptors.

In a press release on Mar. 13, 2017, BioPharmaDIVE™ reported, “There are now 90 cannabinoid drugs in development worldwide, according to a November whitepaper from market analysis firm GBI Research.”

The market for cannabinoids is thus large. The widespread distribution of cannabinoid receptors CB₁ and CB₂, and the apparently wider spread distribution of TRPV₁, a cannabinoid binding Ca channel opens multiple possibilities for cannabinoid based therapies. While some therapeutic applications have been recognized, the wide distribution of receptors and co-molecules that potentiate effects when both are bound to, for example TRPV₁, opens the field for patenting novel therapeutic applications.

Cannabinoids are well-known for their psycho-active effects. The main exogenous cannabinoids that induce sleep are THC, CBD, CBG and CBN. THC is the main psychoactive and also is associated with most of the side effects of marijuana medication. CBD is currently the most medicinal of all the significant cannabinoids and also has the least side effects. CBN is very calming. Good oriental hashish contains large quantities of CBN which provides a reference for medicinal effects. CBG is found in small quantities only in certain strains of cannabis and therefore may be difficult but not impossible to obtain in large quantities. But selective breeding and genetic engineering is available to increase phyto-production of CBG—and indeed other cannabinoids—if increased production is warranted.

CBD differs from THC, and the endocannabinoids anandamide, aka N-arachidonoyl-ethanolamine (ANA or AEA) and 2-arachidonoylglycerol (2AG) in that CBD does not directly bind CB₁ or CB₂, rather CBD heightens endogenous cannabinoid availability by suppressing the FAAH—the enzyme that decomposes AEA.

The endogenous cannabinoids, AEA and 2AG, are factors in cancer pathophysiology through their effects on at least tumor growth and suppression, disease progression, immune response, peritumoral inflammation, nausea and pain. In fact, endocannabinoid profiles are sufficiently aberrant with cancer to produce marked alterations in plasma endocannabinoids of cancer patients.

Intracellular CB₂-dependent signaling pathways involve Gi/o-dependent inhibiting adenylyl cyclase, stimulating MAPK-PI3K pathways and activating de novo ceramide production or COX-2 induction. Tumor growth rates and 2AG levels correlate at the tumor but also in plasma. In the tumor microenvironment 2AG stimulates CB₂ receptor signaling to invading immune cells. This signal may trigger a phenotypic switch from aggressive to tumor-tolerant cells and polarization towards the tumor-helping M2-like macrophages, such as “tumor associated macrophages” (TAMS) which promote tumor invasiveness and metastasis by releasing metalloproteinases and angiogenic factors.

CB₁ and GPR18 receptors in vascular cells when stimulated by cannabinoid agonist(s) signal vasodilation and thereby increase the tumor's blood supply.

Contrary to 2AG, ethanolamide endocannabinoids (eCBs), AEA, O-AEA), oleoylethanol-amide (OEA) and palmitoylethanolamide (PEA) are decreased at the tumor and in plasma, likely because the growing tumor displaces normal cells that produce these eCBs.

OEA levels correlate with the number of metastases in cancer patients, and liver metastases actually cause an increase of OEA plasma levels allowing for tracking and possible LeChatellier feedback. Indeed high OEA concentrations, like those in normal tissue surrounding locally restricted tumors, will inhibit tumor cell migration. However, plasma levels generally do not increase to a level where they exert significant migration inhibition. Local or systemic levels of OEA are assayable as part of a monitoring/treatment relating to metabolic perturbations including cancer tumors.

Beyond the endogenous cannabinoids correlating with and affecting or being affected by cancerous or pre-cancerous cells, endogenous cannabinoid signaling (ECS) is a means of intercellular and intracellular communication used throughout the body. Endogenous cannabinoids affect sleep/wake cycles, temperature regulation), food consumption and fat storage, CNS regulation of autonomic and endocrine functions, reward-driven behavior, gastro-intestinal function, mood and sensory perception. All of these processes are altered in a cyclical manner. ECS has profound repercussions given the intracellular and intercellular communication provided by the cannabinoid system.

ECS in known animal systems uses at least two arachidonate ligands, AEA and 2AG and at least two G-protein-coupled receptors, CB receptor types 1 and 2 (CB₁ and CB₂). Intracellularly at least AEA targets TRPV₁. Both AEA and 2AG are the arachidonate members of larger lipid families: the N-acylethanolamines (NAEs) and the 2-monoacylglycerols (2-MAGs). But with rare exception the other family members do not act as ligands for the CB₁ and CB₂ receptors with little evidence of activity regarding more recently identified, less classical cannabinoid receptors.

Endogenous activation of the CB₁ and CB₂ receptors positively correlates with biosynthesis and negatively correlates with catabolism of both the endocannabinoids AEA and 2AG. AEA is synthesized from N-arachidonyl-phosphatidylethanolamine (N-arachPE), via either a phospholipase D (e.g., NAPE-PLD (N-acyl phosphatidylethanolamine phospholipase D) (or an α,βhydrolase (ABH4) and a glycerophosphodiesterase (GDE1)). At least three enzymes are involved in catabolizing AEA: fatty acid amide hydrolase (FAAH), a lysosome-localized fatty acyl amide hydrolase with an acid optimum (NAAA) and FAAH-2 found in lipid droplets.

AEA has several missions within our cells. It may bind and activate an endocannabinoid receptor such as CB₁ or CB₂. AEA may bind other cannabinoid receptors, e.g., PPAR, TRPV₁, etc. It may be hydrolyzed to AA and ethanolamine. It may be acted on by a cytochrome p450 to form 14,15-epoxyeicosatetraenoic acid ethanolamide (EET-EA)—another CB activator. 12-LOX or 15-LOX can convert AEA to 12- or 15-HETE-EA, another CB₁ and TRPV₁ ligand and EXC₄-EA. And COX-2 can convert AEA to PGEAs.

2AG is synthesized by PLC and diacylglycerol lipase DGL. Monoacylglycerol lipase (MGL), ABHD6 and ABHD12 hydrolyze 2AG.

2AG also has several missions within our cells. It may bind and activate and the endocannabinoid receptor such as CB₁ or CB₂. It may be acted on by cytochrome p450 to form 2-(11,12-epoxyeicsatrienoyl)glycerol (EET-G), another CB activator. 12-LOX or 15-LOX can convert 2AG to 12- or 15-HETE-G, another CB₁ and PPAR ligand. And COX-2 can convert 2AG to PGGs.

Endogenous cannabinoid signaling (ECS) occurs throughout the body in tissues and organs including, but not limited to: central nervous system (CNS) (brain, spinal cord), fat liver, pancreas, immune system, sympathetic nervous system, etc. In the CNS, sympathetic nervous system, sensory nerves and enteric nerves, CB₁ receptor on the presynaptic side inhibits neurotransmitter release thus asserting a modulatory influence. Stress down-regulates CB₁ receptors at least in the hippocampus where AD damage is noticed early, while CB₂ expression is responsive to activation states of macrophages. CB₂ receptors are predominantly found in macrophages and other cells of the immune system with highest concentration in the spleen. The responsiveness of CB receptors to changing conditions is indicative of their integral role in the body's responses to external events.

Cannabinoid pathways are also interactive with internal events. Cannabinoid levels and expression of CB receptors present diurnal variation in a tissue dependent pattern. Administering exogenous cannabinoids can bring on sleep, and affects body temperature and other hypothalamic/pituitary axis functions, feeding, hippocampal consolidation, etc. In addition to the hippocampus, hypothalamus, striatum, prefrontal cortex and pons, of the CNS, hepatic, pancreatic and adipose tissues all show diurnal responsiveness patterns to cannabinoid manipulation. Cannabinoid effects are profound in peripheral tissues with CB₁ expression extensive in organs or tissues including, but not limited to: liver, capillary wall, gonads, heart, liver, uterus, renal/adrenal, etc. The wide distribution of these receptors renders each of: the receptors, transcription initiation events, RNA control, Translation, cellular transport, mitochondrial binding, synthesis and hydrolysis on endocannabinoids susceptible to adverse outcomes during a body's or a cell's opportunistic adaptations to life's stresses. The wide distribution also renders the synthetic and catabolic pathways for the receptors, the cannabinoids, the synthetic and hydrolytic pathway enzymes, the intracellular trafficking expulsion, recognition and uptake of these endo cannabinoids valued targets for ameliorating the metabolic inefficiencies.

CB₁ receptor activity supports slow wave and rapid eye movement sleep stages. AEA supports healthy sleep. Together CB₁ and AEA partner to support healthful sleep habits. In compensation for sleep deprivation, CB₁ expression increases in the pons, possibly to depress activity levels.

AEA when active in the nucleus acumbens and ventral hippocampus stimulates oxytocin, the feel good hormone associated with successful social interaction. Oxytocin both promotes social behavior and is released in response to successful bonding events. In peripheral tissues, oxytocin under the influence of cannabinoids like AEA is active in several significant and beneficial functions, such as promoting uterine contraction for childbirth and stimulating bone growth following cannabinoid stimulation of osteoblasts. 2AG does not have similar effect. This differential sensitivity permits a selective bias in promoting oxytocin effects over other cannabinoid influences. Inhibiting FAAH can augment or substitute for AEA or AEA mimetic enhancement of these activities. NADA, N-benzyl-9Z-octadecenamide, N-(3-methoxybenzyl)-9Zoctadecanamide, N-pyridine-9Z-octadecenamide, N-(3-methoxybenzyl)-(9Z,12Z)-octadecadienamide, N-benzy-(9Z,12Z)-octadecadienamide, N-benzy-(9Z,12Z,15Z)-octadecatrienamide, N-(3-methozybenzyl)-(9Z,12Z,15Z)-octadecatrienamide are examples of FAAH inhibitors. Any of the known FAAH inhibitors as well as FAAH inhibitors extracted from fungi and other microbes are capable of use for enhancing AEA effect.

AEA appears to be released selectively in response to socialization. 2AG and OEA are not similarly released in these situations, though the membrane lipids that act as endocannabinoid sinks contain precursor molecules for them. Endocannabinoids are “on demand” signal molecules produced under a “just-in-time” inventory system. Unlike classic neurotransmitter substances and hormones stored in vesicles for massive release in the future, the minimal synthetic pathways required for active cannabinoids allows them to be harvested from common cell membrane components.

Fatty acid synthesis in the liver is increased when CB₁ receptors are activated there. Obesity results in mice lacking hepatic CB₁ receptor. CB₁ is a critical piece of the cell's metabolism with systemic ramifications.

The physiological effects of cannabinoids are mediated by two well-known high-affinity cannabinoid receptors, CB₁ and CB₂, spanning the plasma membrane. Both CB₁ and CB₂ interact with the inhibitory G-protein α-subunit Gᵢ and G₀. When activated adenylate cyclase is inhibited, decreasing production of second messenger molecule cyclic AMP (cAMP), and increasing activation of mitogen activated protein kinases (MAPK). But, under some conditions in the presence of certain agonists, CB₁ and CB₂ receptors will couple with Gₛ and G_{q11}. Furthermore, CB₁ and CB₂ activations also appear to have distinct downstream effects on several other intracellular signal transduction pathways including, but not limited to: opening potassium (K⁺ or K for short) ion channels, closing calcium (Ca²⁺ or Ca for short) channels, through decreased protein kinase A and C (PKA and PKC) activity, and affect MAPKs like Raf-1, ERK, JNK, p38, c-fos, and c-jun, depending on the cell type. CB₁ receptors modulate ion channels on presynaptic neurons, inhibiting N-and P/Q-type calcium channels and slowing glutamate release, stimulating inwardly rectifying K channels and enhancing the activation of the A-type K channel. Although the cannabinoid receptors are probably best characterized for their activities in the CNS, their distribution is much broader. For example, prostate and lymph cancers frequently express high levels of CB₁. Similarly, gastro-intestinal tissues: e.g., appendix, colon duodenum, esophagus, salivary gland, small intestine, and stomach; adrenal, kidney, urinary bladder, thyroid, ovary, testis, spleen lung, lymph gall bladder, vascular endothelial cells and fat actively express this receptor. CB₂ is most prolifically expressed in hematopoietic and immune tissue, e.g., macrophages, spleen, tonsils, thymus leucocytes, lung and testes. 2AG has a higher maximal effect on CB₂ receptors than AEA.

As GPCRs, CBs have versatile signaling capabilities through coupling with disparate G proteins, oligomerization with like or other GPCRs for precise signaling, sensitization and desensitization modules, interaction with modulator proteins and interaction with lipid raft domains.

CB₁ is concentrated in the brain and central nervous system, but is expressed in many peripheral tissues. CB₁ is a factor in a broad range of psycho-physiological functions, including, but not limited to: emotional learning, stress adaption, and fear extinction. CB₁, when functioning normally, deactivates traumatic memories and drives the process of forgetting. Proper CB₁ activity is important during childbirth and other necessary stress situations. However, when CB₁ is not properly activated, e.g., poor ω-fat diet has reduced AEA availability, PTSD and similar outcomes can be particularly severe.

Besides compromised diet, alcoholism and chronic stresses are other sources of endocannabinoid deficits. Yet, cannabinoid receptor signaling has been identified as a key modulator of adaptation to stress.

CB₁ stimulation activates adenylyl cyclase indicating the activation cascade includes Gs, but other Gₑₙₜᵢₜᵢₑₛ can be involved. For example, CB₁-mediated stimulation of cAMP formation can result from Gβγ dimers released from Gᵢ proteins. Ca²⁺ flux following CB₁ stimulation occurs and increases in Ca²⁺ levels inside several, but not all cell types. Ca²⁺ can be mediated by Gi/o proteins through the β2 isoform of PLC and the Gβγ subunits. These differential effects of AEA on different downstream paths in different cell types permit selective activation, and when coordinated with β-arrestins, selective modulatory effects. These cell-type-specific differences in the mechanism of CB₁-mediated Ca²⁺ induced cascade effects allow these cells to be selectively modulated.

But GPR55 function, is predominantly through Ca²⁺ signaling depending on ligand rather than cell type. GPR55 elicits Ca²⁺ flux activation when stimulated by THC, AEA, methanandamide and JWH015 (selective for CB₂), but not by 2AG, PEA, CP 55 940, or O-AEA. Different ligands can thus be used to activate these different cannabinoid controlled pathways in selected different cell types.

ω-3 and ω-6 fats are essential for endocannabinoid production. Muscle stimulus, e.g., exercise or stretching support endocannabinoid levels. Pepper, a TRPV₁ ligand, and terpenes boost endocannabinoid activity. NO induces cannabinoid receptor transcription. Sunlight (uv radiation) and vitamin D support this effect. Folate, resveratrol, capsaicin, caffeine, pain relievers (stress reducers), vitamin A and supplements: including, but not limited to: flavones, e.g., 7-hydroxyflavone and 3,7-dihydroxyflavone, genistein, agmatine, kava, TEA, inositol, honokiol, etc. help maintain healthy cannabinoid levels.

A lower ω-6/ω-3 ratio is one factor for ameliorating cannabinoid related physiological deficits. Modifying this ratio by manipulating diet, adjusting temperature, modifying (increasing or decreasing) metabolisms of organs, tissues or cells that act as sinks for theses lipids may be considered especially in the management of obesity and as part of balancing cannabinoid influences on the body stasis and additional metabolic supplements.

CB₁ receptors have been shown to stimulate MAP kinase. The MAP kinase pathways (MAPK) operate a key switchpoint for regulating numerous cell proliferation and survival functions including, but not limited to: cell growth, transformation and apoptosis. MAPK's activation is classically associated with an initiation activation of a tyrosine kinase-linked receptor. This activates the intracellular G protein, Ras, and tees up a signaling cascade beginning with activation of the serine/threonine kinase, Raf (MAP kinase kinase kinase). Raf activates MAP kinase kinase (MEK) catalyzing phosphorylation and activation of MAP kinase, which then phosphorylates perhaps, depending on intracellular conditions, scores of cytoplasmic and nuclear proteins including numerous transcription factors (promotors, repressors, enhancers, (de)acetylases, (de)methylases, transferases, kinases, phosphatases, (de)ubiquitinases, etc. The four best characterized MAP kinase families (ERK1/2, JNK, p38, and ERK5 proteins) are understood to coordinate cellular responses by phosphorylating and regulating the activity of multiple substrate proteins in pathways involved in transcription, translation, and changes in cellular architecture.

MAPKs have a multitude of effects. Since transcription factors are common products of the cascade, entire functions, including differentiation progression and cell division are under the purview of MAPKs.

As such several large and small molecule interventions for specific MAPKs and MAPK activities have been developed. Well-characterized specific MAPK inhibitors include, but are not limited to: IRESSA® (gefitinib), TARCEVA® (erlotinib), TYKERB® (lapatinib), Sutent® (sunitinib), NEXAVAR® (sorafenib), TASIGNA® (nilotinib), SPRYCEL® (dasatinib), imatinib, GLEEVEC® or GLIVEC® (imatinib, tested as a mesylate), ZARNESTRA® (tipifarnib), NEXAVAR® (sorafenib), U0126 (a highly selective inhibitor of both MEK1 and MEK2), PD184352 (an ATP non-competitive MEK1/2 inhibitor), Selumetinib (AZD6244, potent and selective inhibitor of MEK1/2 with tumor-suppressive activity), BIX02188 (selective inhibitor of MEK5, but does not MEK1, MEK2, ERK2, and JNK2), BIX02189 (MEK5 and ERK5 (BMK1) kinase inhibitor), SB203580 (p38 MAP kinase inhibitor—also inhibits Akt), SB202190 (highly selective, potent and cell permeable inhibitor of p38 MAP kinase), Doramapimod (BIRB-796, pan-p38 MAPK inhibitor), PD98059 (highly selective inhibitor of MEK1 activation and the MAP kinase cascade), etc., which selectively or preferentially interact with EGFR, VEGFR, VEGFR, PDGFR, PDGFR, Bcr-Abl, Bcr-Abl/c-Src, Bcr-Abl/c-SCT/ c-Kit/PDGFR, Ras, MEK1/2, MEK1/2, MEK1/2, MEK5, p38α, p38γ, p38δ, MEK1, respectively. Additional antibodies and antibody derivatives have been and can readily been made and matched for different affinities/specificities and binding sites including, but not limited to: recognition sites, ATP binding sites, non-catalytic sites, co-factor sites, etc. on these and other MAPK targets. For example, since MAPKs share a common evolutionary route, related MAPK families can be targeted by targeting shared sequence areas. Analogues of the small molecules are also readily available to the skilled artisan and can be selected based on ease of manufacture/delivery, solubility, stability, tagging, multiple interactive sites, minor changes in architecture, etc. Other less chemically analogous inhibitors can be designed, tested and manufactured by the skilled artisan using known structure function relationships, high-throughput screening and 3-D architecture of the targets available for most MAPKs through crystallography and mathematical modelling. Kimberly Burkhard and Paul Shapiro in a 2013 publication provide a discussion of methods, history and development of several compounds for laboratory and/or therapeutic use.

The two most studied endogenous cannabinoids, AEA (N-arachidonoylethanolamine), aka ANA (anandamide), and 2AG (2-arachidonoylglycerol), have notable effects on our moods, appetite, pain sensation, inflammation responses, and memory.

These endocannabinoids are lipids, derived primarily from the ω-6 fatty acids. Their concentrations are limited by dietary intake of ω-6 and ω-3 fatty acids; and by availability and activity of the synthetic and catabolic enzymes involved. A diet with a high ω-6/ω-3 ratio facilitates an increase in the endocannabinoid signaling and related mediators, which may lead to an increased inflammatory state, energy homeostasis, mood, blood pressure, apoptosis, etc. depending on the state of the organism or cell and how the ω fats are processed. Greater amounts of ω-3 fatty acids tend to increase insulin sensitivity and to control body fat. Deficiencies of ω-3 oils and related probiotics, low vitamin D levels, and other nutritional imbalances may contribute to an individual's mitochondrial dysfunction. Many prescription pharmaceuticals, artificial sweeteners, and food additives can feed similar outcomes. The ω fats, their metabolites and modulations of major metabolic pathways are important control factors for structure and functions of most body tissues.

Anandamide's (AEA's) importance is emphasized by the alternative pathways we have available to synthesize it. The first recognized pathway diverts N-arachidonoyl phosphatidyl ethanolamine from membrane synthesis using a phospholipase D (NAPE-PLD) to make AEA. Increasing NAPE-PLD by stimulating relevant transcription factors, maintain mRNA function, or other means serves to augment AEA levels. Similarly, NAPE-PLD knockdown, e.g., inhibiting or blocking transcription, or degrading mRNA function can decrease AEA. But our cells have back-up pathways to ensure AEA availability when cell conditions call for it. Some tissues express a phospholipase A₂ that converts NAPE to 2-lyso-NAPE which is processed to AEA. In tissues lacking the phospholipase A₂, a pathway using α,β-hydrolase 4 (Abhd4) produces AEA when conditions warrant. An additional pathway identified in cells responding to endotoxin involves a phospholipase C and tyrosine phosphatase to produce the AEA.

Similarly, 2-arachidonoylglycerol (2AG), believed to be the most abundant endocannabinoid, is produced by several mechanisms several of which produce 2AG for further metabolism to other molecules. Most 2AG is synthesized from membrane phospholipids via sequential activation of a phospholipase Cβ and a diacylglycerol lipase, chiefly DAGLα and DAGLβ. 2AG can also be synthesized by dephosphorylation of arachidonoyl-LPA. Another alternative or backup pathway involves Phospholipase A1 and a lysophospholipase C.

The endocannabinoid system is involved in regulation of energy balance. Sustained hyperactivity of the endocannabinoid system contributes to obesity. Arachidonic Acid (AA) is a precursor for biosynthesis of 2AG and AEA. Increasing the precursor pool of AA leads to excessive endocannabinoid signaling of paths for weight gain and development of a metabolic profile associated with obesity. Endocannabinoids bind to and activate endogenous cannabinoid CB₁ and CB₂ receptors in brain, liver, adipose tissue, immune system and the gastrointestinal tract. Activation of CB₁ receptors in the hypothalamus encourages increased appetite and food intake. Endocannabinoids selectively enhance sweet taste, which in the current highly palatable food supply stimulate food intake. The endocannabinoid system functions in concert with other systems regulating food intake and energy balance, and is regulated by leptin, insulin, ghrelin, cholecystokinin, and other signals. Targeting the endocannabinoid system has been a strategy for weight loss diet pills attempted without success by major pharmaceutical companies. Randomized controlled clinical trials in overweight or obese humans showed that CB₁ receptor antagonists such as rimonabant led to significant weight loss after one year of treatment. However, the drug was withdrawn from the market due to severe side effects that led to increased risk of anxiety, depression, and suicide. CB₁ inhibition by these drug candidates theoretically could induce these effects by decreasing oxytocin.

CB₁ also shows a high degree of basal activity absent cannabinoid activation. It might be classified as a constitutively active receptor. However, since not all cannabinoid activators have been identified and there is a significant probability that at least one endogenous activator, e.g., 2AG, is often constitutively present; the constant state of activity might alternatively be classified as a constitutively present ligand. For example, the endocannabinoid, 2AG, when assayed is found to be present in concentrations close to the Kd of CB₁. Diacylglycerol lipases (DAGL), enzymes synthesizing 2AG, are present in most tissues. At least this endocannabinoid may be sufficient to constitutively activate the receptor.

In neural tissue, 2AG is degraded both pre- and post-synaptically. In most tissues 2AG metabolism can occur in multiple cell compartments with profound results. As an example, COX-2 produces prostaglandin glycerol esters from 2AG. These esters tend to be opposite in activity to 2AG, essentially switching off or switching back the effect that 2AG had.

Other CB₁ antagonists include selective CB₁ receptor antagonists SR141716 (SR1), SR141716A (SR1A) AM251 and AM281 CB₁ and CB₂ receptor antagonists, SR141716A and SR144528, respectively.

Although cannabinoid receptors maintain important functions, sometimes these functions must be down-modulated. Other than simply dialing back receptor ligand availability which may be imprecise given all the cell membranes, intracellular and extracellular with cannabinoid activation, tools within the cell to dial back cannabinolic activity would be expected. Indeed, organisms have such tools at their disposals.

Repeated exposure to THC leads to tolerance and dependence.

One such dial back tool is exemplified in the β-Arrestin system. β-Arrestins were identified as major proteins involved in these processes. Expression and binding to CB₁ is not affected by presence or lack of β-Arrestins in mouse models indicating downstream pathway effects. β-Arrestins after phosphorylation to activate them bind GPR and inactivate them by internalization. CB₁ binding then loses the ability to interact with its associated GPR cascades.

β-Arrestins are selective. They will bind differentially to different GPRs and not restricted to GPRs, but can bind and modulate activity of many transmembrane proteins. β-Arrestins can be used to desensitize at least CB₁ and GPR cannabinoid receptors. The mechanism of differential selective binding by β-Arrestins is not fully characterized. However, given evidence that the β-Arrestins downstream antagonism of GPR pathways is receptor specific and often ligand specific, the recruitment of specifically acting β-Arrestins for antagonistic action with respect to specific ligands is achievable with prolonged exposure to the target ligand. This need not risk in vivo complications as cells in culture can be stimulated to produce the specific β-Arrestins for the desired ligands and target receptors.

Ligand-induced signaling from G protein-coupled receptors (GPRs) can differentially stabilize receptors into multiple signaling conformations resulting in pluridimensional efficacies resulting in functional selectivity or biased signaling. When activated by ligand, GPCRs undergo conformational changes that activate heterotrimeric G proteins and their effectors such as adenylyl cyclase. GPR kinases (GRKs) differentially phosphorylate GPRs, generating specific phosphorylation patterns depending on the ligand that are subsequently recognized by β-arrestins.

β-Arrestins serve two functions. The more obvious is negative regulation of heterotrimeric G protein signaling during receptor desensitization and internalization. They also act as signaling scaffolds. When regulating receptor activity, β-Arrestins block G protein signaling and recruit components of the endocytic machinery to initiate receptor endocytosis. As a signaling scaffold the phosphorylation pattern appears important, but understanding of β-Arrestin-mediated signaling is still rudimentary. However, controlled culture can produce relevant patterns of phosphorylation for specific ligands and receptors.

While evidence is clear that cannabinoids have important cytoprotective functions, especially in neuro-tissue, endogenous cannabinoid and CB₁ agonists can also be put to use in inducing cell death through apoptosis to protect the organism at large. This available process involves multiple paths probably through multiple cannabinoid receptors, e.g., CB₁ and TRPV₁, possibly 5-HT_{1A}. In situations, for example, an immune response, where apoptosis is beneficial, endocannabinoids can be put to good use by stimulating ROS production, caspace activation and p38 and JNK MAPK pathways to elicit an effective apoptotic response. Selective targeting is essential though because the body cannot survive when all cells spontaneously apoptose. Selective delivery and dosage control are thus important for optimal control of cannabinoid effects. Identification and targeting tissues and cells to be treated with a selected cannabinoid to achieve the desired effect is one feature of this invention. CB₁ activation when coupled through Gi/o frequently operates through ERK1/2, JNK1, JNK2, and p38.

The p38 family includes four members, p38α, p38β, p38γ and p38δ, with greater than 50% homology between them. P38s are well-known for their contributions in immune and inflammatory responses. p38s' involvement in intracellular transport and compartmentalization of functions within the cell is of vital importance to cell health and survival.

Cannabinoid receptor agonists and/or antagonists may be used for treating a variety of diseases including inflammatory pain, reflex sympathetic dystrophy/causalgia, cataract, macular degeneration, peripheral neuropathy, entrapment neuropathy, complex regional pain syndrome, nociceptive pain, neuropathic pain, fibromyalgia, scleroderma, chronic low back pain, visceral pain, acute cerebral ischemia, pain, chronic pain, psoriasis, eczema, acute pain, post herpetic neuralgia (PHN), neuropathies, neuralgia, diabetic neuropathy, HIV-related neuropathy, nerve injury, ocular pain, headaches of various etiologies—including migraine, stroke, acute herpes zoster (shingles), pain-related disorders such as tactile allodynia and hyperalgesia, rheumatoid arthritic pain, osteoarthritic pain, back pain, cancer pain, dental pain, muscular pain, mastalgia, pain resulting from dermal injuries, fibromyalgia, neuritis, sciatica, inflammation, neurodegenerative disease, cough, broncho-constriction, irritable bowel syndrome (IBS), inflammatory bowel disease (IBD), colitis, cerebrovascular ischemia, emesis such as cancer chemotherapy-induced emesis, rheumatoid arthritis, Crohn's disease, ulcerative colitis, asthma, dermatitis, seasonal allergic rhinitis, gastroesophageal reflux disease (GERD), constipation, diarrhea, functional gastrointestinal disorders, irritable bowel syndrome, cutaneous T cell lymphoma, multiple sclerosis, osteoarthritis, psoriasis, systemic lupus erythematosus, diabetes, glaucoma, osteoporosis, glomerulonephritis, renal ischemia, nephritis, hepatitis, cerebral stroke, vasculitis, myocardial infarction, cerebral ischemia, reversible airway obstruction, adult respiratory disease syndrome, chronic obstructive pulmonary disease (COPD), cryptogenic fibrosing alveolitis, and bronchitis.

Cannabinoids have numerous physiological effects believed to be mediated through their G protein-coupled receptors CB₁ and CB₂, the ion channel TRPV₁, GPR55, 5-HT_{1A}, GPR119, PPAR and their corresponding cascading systems, many cannabinoid-evoked actions are attributable to one or a combination of these specific receptor targets.

Anandamide (ANA), aka N-arachidonoylethanolamine (AEA), is a fatty acid neurotransmitter metabolized from eicosatetraenoic acid (arachidonic acid), an essential ω-6 polyunsaturated fatty acid metabolite. In mammals, it is deactivated primarily by the fatty acid amide hydrolase (FAAH) enzyme, which converts anandamide into ethanolamine and arachidonic acid. Inhibiting FAAH can lead to elevated anandamide levels locally or systemically.

AEA is involved in regulating feeding behavior, as well as neural generation of motivation and pleasure. AEA injected directly into the forebrain reward-related brain structure nucleus accumbens enhances pleasurable responses and enhances food intake. Runner's high may be mediated by anandamide. AEA is biosynthesized from N-arachidonoyl phosphatidyl-ethanolamine (NAPE) which arises by transfer of arachidonic acid from lecithin to the free amine of cephalin through an N-acyltransferase enzyme. Endogenous anandamide is present at very low levels and has a very short half-life due to the action of FAAH, which breaks it down into free arachidonic acid and ethanolamine.

Acetaminophen is metabolically combined with arachidonic acid by FAAH to form AM404 which is a potent agonist at the TRPV₁ vanilloid receptor, a weak agonist at both CB₁ and CB₂ receptors, and an inhibitor of AEA reuptake. AM404 is a synthetic anandamide analogue replacing the ethanol amine of AEA with a phenolamine. When AM404 is present slowing AEA reabsorption, AEA levels in the body and brain are elevated. In other words, acetaminophen acts as a pro-drug for a cannabimimetic metabolite. This cannabimimetic action is partially or possibly fully responsible for acetaminophen's analgesic effects. AEA is also an agonist of TRPV₁.

TRPV₁ is expressed cell's internal membranes, e.g., in the ER, the nucleus and mitochondrion. TRPV₁ when activated provides a pore through which Ca⁺⁺ flows in accordance with its concentration gradient. Protein kinase A (PKA), protein kinase C (PKC), Ca⁺⁺/calmodulin-dependent kinase II (CaMKII), and Src kinase modulate TRPV₁'s activity by lowering its activation thresholds or time remaining open. TRPV₁'s is modulated by multiple intracellular signals, including, but not limited to: γ-amino butyric acid A-type (GABAA) receptor associated protein (GABARAP), E3-ubiquitin ligase myc-binding protein-2; cyclin-dependent kinase 5 (Cdk5), kinesin-3 family member 13B (KIF 13B), etc. TRPV₁ is sensitized by nerve growth factor (NGF), a protein with angio-support responsibilities, especially in joints, and is translocated to the plasma membrane in response to insulin. Capsazepine and ruthenium red are the most studied TRPV₁ antagonists. Activation of TRPV₁ has a cooling influence on the organism as a whole. Systemically, the sweat and vasodilatory responses following eating hot peppers embodies this effect. TRPV₁ activation also decreases superoxide production by decreasing the resistance of electron flow down the respiratory chain. TRPV₁ activation has been associated with reduced insulin, glucose, fats in the plasma, and microbleeding events and with increased glucagon like peptide activity and brown fat metabolism.

While AEA and capsaicin have similar affinities for TRPV₁, AEA has a significantly lower potency. The agonist binding sites on TRPV₁s are intracellular. This requires AEA to cross the plasma membrane to have effect. Though in many observations AEA appears to cross plasma membranes and synthetic membranes without a facilitator under some conditions transport AEA transport involves a dedicated carrier, a membrane crossing facilitator substance responsive to NO). This facilitator is given the functional name as AMT (AEA membrane transporter). Since AMT is activated by NO, AEA induced vasodilation is reduced by NO synthase inhibitors. The NO affinity and its facilitation is useful for abetting transport and targeting to specific membranes and orienting the endocannabinoid therein.

Then, once in the cell AEA is catabolized by FAAH to arachidonic acid and ethanolamide. Cyclooxygenase-2, lipoxygenase and P450 enzymes in some tissues are available to convert AEA and products to bioactive compounds. PEA (palmitoylethanolamide) inhibits AEA catabolism. PEA is often coproduced with AEA.

Another potentiator of TRPV₁ is its phosphorylation, e.g., by protein kinases A and C. This contrasts with PKC phosphorylation of CB₁ which block cannabinoid activation.

The near universal distribution of TRPV₁ may be related to the pervasiveness of Ca channels throughout the cell, in plasma membranes as well as the membranes of multiple organelles. The ability of many influences, including, but not limited to: capsaicin, allyl isothiocyanate, temperature, H⁺, N-arachidonoyl-dopamine, anandamide, N-oleyl-dopamine, etc. speaks to the importance of this receptor-pore. Activation can be quite drastic, for example, beyond a threshold, activation of ER TRPV₁ cooperates with mitochondria to carry out apoptosis.

CBD is a potent activator of TRPV₁ as is AEA and the acetaminophen metabolite AM404. AM1172, a structurally similar isomer to AM404 is another and more potent TRPV₁ activator. AM1172, N-(5Z,8Z,11Z,14Z)-5,8,11,14-eicosatetraen-1-yl-4-hydroxybenzamide, is resistant to FAAH hydrolysis and has a longer half-life than AM404. Whereas AEA and AM404 are formed by condensing arachidonic acid, a polyunsaturated acid, with an alcohol amine (ethanolamine and phenolamine, respectively), AM1172 is the condensation product of a polyunsaturated amine (with the same polyunsaturated chain as arachidonic acid) with an acid alcohol (4-hydroxybenzoic acid). The '1172 isomer simply reverses the order of the amide and keto groups off the phenol. AM1172 is an inhibitor of FAAH. AM 1172 is an activator of CB₁ and CB₂.

It may be a matter of semantics relating to TRPV₁ and its ligands. Given that many cannabinoids and their synthetic analogues bind and activate/inhibit TRPV₁, TRPV₁ would properly be considered a cannabinoid receptor. In this case, other ligands for TRPV₁ should also properly be recognized as cannabinoids. N-arachidonoyldopamine, an endocannabinoid found in cerebral spinal fluid and recognized to bind TRPV₁ in human CNS cells, chemically is a capsaicin analogue and thus capable of modulatory influence through capsaicin and/or its analogues.

Prostaglandin E2, a mediator of inflammation in diseases such as rheumatoid arthritis and osteoarthritis is a metabolite of AA. AEA and 2AG are oxidized by COX-2 into PG-ethanolamide (PG-EA) and PG-glycerol (PG-G), respectively. Alternatively, FAAH 1 and FAAH2 simply hydrolyze AEA into AA and ethanolamine.

It has been suggested that AM1172 could be developed into a drug that would increase the brain's anandamide levels to help treat anxiety and depression. Black pepper contains an alkaloid, Guineensine, which appears to be a relatively potent AEA reuptake inhibitor, thus increasing its physiological effects. Low doses have an anxiolytic effect. High doses in mice cause hippocampus death. The amount of AEA in cacao is about 0.5 μg per gram.

Human life involves multiple trillions of cell divisions during a normal lifetime. And each cell division involves thousands of coordinated events, including maintenance of organelles that may individually require several thousands of biochemical creations within and without the organelle. It's amazing to consider that all these multi-trillions of steps can coordinate sufficiently to bring us to adulthood and beyond. However, as every adult knows and feels, not all the reactions within our bodies are as optimal as they once were. The biochemical events in our bodies today are not the more vigorous life enhancing events we experienced in our past. As we progress through our lives our cells respond to changes in the physical and chemical properties of the environment by continuously altering many cellular functions with outcomes critical to cell and organism survival, proliferation, metabolic rate, interaction with other cells, and organisms, temperature control, feeding behaviors, activities, etc. We also may change our environment through our responses to it. The environmental changes require balancing on our end to maintain homeostasis, optimal functioning and ultimately continued survival. Environmental changes that challenge us as organisms or our individual cells may include nutrient type and availability, temperature, position, growth factors, ions, cytokines, toxins, light, oxygen, physical and/or emotional stimulation, etc.

When metabolism is functioning optimally, the cell has appropriate substrate available for each beneficial pathway. For each such pathway the cell delivers appropriate substrate, the substrate is processed and the products are delivered to the next step in that pathway. When an atypical result occurs, it may be impossible for the next reaction to occur. This product may be secreted from the cell, may be used in a different pathway, may bind or otherwise interfere with another molecule in the cell, may be degraded by scavenging actions within the cell, or may just float around getting in the way. Usually the cell has a means of correcting or discarding metabolic errors. But often when an unexpected substrate (perhaps a drug or toxin or just an unaccustomed food) or an abnormal amount of substrate presents, the cell will switch its biochemical machinery in response to the stress, perhaps activating a kinase, inducing transcription of an enzyme or receptor, tagging an enzyme for recycling, or epigenetically altering the activity of genetic material. Sometimes these changes are not easily reversible but managed in the cell. Sometimes these may lead to cell death through initiation of apoptosis. Generally, the adaptations trigger changes in related pathways which may produce a small or large imbalance of the cell's original metabolic status.

In responding to such stresses, our cells activate pathways opportunistic at that moment for those conditions both intracellular and extracellular. These opportunistic compensations will generally include modifications in several subfamilies of MAPKs including, but not limited to: ERK1/2, ERK5, JNKs and p38s. With each such stress and opportunistic response our cells will have accumulated baggage—changes from the more optimal balance the cells originally had before stresses forced changes in their structures and metabolisms. Accordingly, for each cell and each instance the response left to itself will take its own course. One feature of this invention is to take into account a general condition, but also to be cognizant of significant differences from other cells or from a normal healthful situation a single cell or a group of cells may present. JNKs and p38s are of particular attention in the present invention which is predicated on stress reversal and relief. These subfamilies together share the nickname SAPKs (Stress-Activated Protein Kinases).

MAPKs are difficult to activate in that there is a cumbersome pathway to MAPK activation. MAKPs are phosphorylated by a MAPK kinase (MAPKK) which itself must have been phosphorylated by a MAPK kinase kinase (MAPKKK). Only a single enzyme, a MAPK phosphatase is sufficient to dephosphorylate MAPK, deactivate the MAPK and cease feeding the cascade. Activation however, is more intricate wherein the MAPKK phosphorylates it MAPK in two specific positions—a tyrosine (Y) and a Ser/Thr (S/T) residue. The various MAPKKKs and MAPKKs have their respective specificities; for example, MKK3 and MKK6 do not activate JNK, ERK1/2, only p38 MAPKs. And p38α has specific alternative phosphorylating kinases in specifically differentiated cell types. E.g., when TAB1, one such alternative kinase, activates p38α in a cardiomyocyte, the p38 is restricted to the cytosol and only get proteins there.

P38 acts as a switch in that its actions are short lived. For example, PTP (Y phosphatase) and PP2C (S/T phosphatase) are 2 deactivators which appear to be constitutively expressed. Although the actions are short lived their after effects are carried through in their cascading after effects. When activated, for example p38α and p38β phosphorylate and activate transcription factors including, but not limited to: MEF2, SAP1, ATF2, CHOP, PGC-1, p53, USF1, STAT-1, NFATp, CDX3, etc. and kinases including, but not limited to: MAPKAP-K2/MAPKAP-K3 MNK1/MNK2, MSK1/MSK2, etc. that phosphorylate additional proteins down the cascade. The results of all these activities will have removed substrate and added product, possibly a methylated DNA molecule whose reaction may never be reversed. These baggages are to be taken into account when restoring the cells/organism's metabolism. While p38γ and p38δ typically phosphorylate substrates including, but not limited to: ATF2, Elk-1, SAP1, etc. and p38δ appears to specialize in phosphorylating its proprietary targets including, but not limited to: stathim, a cytoskeletal protein, MAPτ (microtubule associated protein tau), eukaryotic elongation factor 2 (eEF2) kinase, etc.

Only p38α activity controls chemotaxis in response to cytokines including, but not limited to: VEGF, fMLP, C5a, PDGF, TGFβ and IL-1β. p38β, p38γ and p38δ do not participate.

Virodhamine (O-arachidonoyl ethanolamine; O-AEA) is an endocannabinoid and a nonclassic eicosanoid, derived from AA. However, O-AEA is arachidonic acid and ethanolamine joined by an ester linkage, the opposite of the amide linkage found in anandamide. It antagonizes CB₁ and agonizes CB₂. Concentrations of in the human hippocampus are similar to those of anandamide, but they are 2- to 9-fold higher in peripheral tissues that express CB2. O-AEA lowers body temperature in mice, demonstrating cannabinoid activity in vivo

Oleamide is an organic compound with the formula CH₃(CH₂)₇CH═CH(CH₂)₇C(O)NH₂. Oleamide has a variety of industrial uses including as a slip agent, a lubricant, and a corrosion inhibitor. It is the amide derived from the fatty acid oleic acid. It is a colorless waxy solid. It is naturally occurring. Oleamide is biosynthesized from N-oleoylglycine. Oleamide's bioactivity was confirmed when it was identified as a results-influencing contaminant diffusing from polypropylene plastics in laboratory experiments. Oleamide is a sleep inducing compound which is naturally released into the CNS in response to sleep deprivation. Oleamide also has thermoregulatory activity in mammals as well as an inhibitory effect on gap junction communication. Oleamide through an uncharacterized pathway blocks cell-to cell transfers, e.g., of apoptotic stimuli.

A mitochondrial membrane protein and enzyme in the electron transport chain, cytochrome C acts with H₂O₂ as a cofactor to amidate oleoyl-CoA. Cytochrome C activation in the early stages of apoptosis along with superoxide dismutase activity producing H₂O₂ may act as a negative feedback interrupting an apoptotic wave transgressing through a mass of cells.

An alternate synthetic route also involves cytochrome C acting to form N-oleoylglycine which is converted by peptidylglycine α-amidating monooxygenase to oleamide. This reaction pathway appears predominant in oleamide's functions as a sleep inducer.

N-Arachidonoyl dopamine (NADA) is an endocannabinoid that acts as an agonist on CB₁ receptor and TRPV₁. NADA has anti-oxidant, anti-allergic and anti-inflammatory functions, and promotes vasorelaxation in blood vessels. NADA activates cannabinoid CB₁ receptors, but not dopamine Dl and D2 receptors. NADA is an FAAH inhibitor and through this activity has the effect of enhancing effects of other cannabinoids.

Fatty acid amide hydrolase (FAAH) is an important enzyme for deactivating endogenous cannabinoids. Its wide selectivity of substrates including, but not limited to: AEA, oleamide, 2AG, O-AEA, many ω-fatty ethanolamines, etc. FAAH has been a target pursued by several pharmaceutical companies with published reports referencing compounds including, but not limited to: AM374, palmitylsulfonyl fluoride, ARN2508, BIA 10-2474, BMS-469908, CAY-10402, JNJ-245, JNJ-1661010, JNJ-28833155, JNJ-40413269, JNJ-42119779, JNJ-42165279, LY-2183240, Cannabidiol, MK-3168, MK-4409, MM-433593, OL-92, OL-135, PF-622, PF-750, PF-3845, PF-04457845, PF-04862853, RN-450, SA-47, SA-73, SSR-411298, ST-4068, TK-25, URB524, URB597 (KDS-4103), URB694, URB937, VER-156084, V-158866, etc.

Endocannabinoid enhancers (eCBE) are available to enhance the activity of the endocannabinoid system by increasing extracellular concentrations of endocannabinoids. These include, but are not limited to: FAAH inhibitors, monoacylglycerol lipase (MAGL) inhibitors, endocannabinoid transporter (eCBT) inhibitors, etc., including AM404, the acetaminophen active agent.

A diet with a high ω-6/ω-3 ratio causes an increase in endocannabinoid signaling and related mediators, which lead to an increased inflammatory state, energy homeostasis, and moodiness. Animal experiments reveal that a diet high in ω-6 acid leads to decreased insulin sensitivity in muscle and promotes fat accumulation in adipose tissue. Diets high in ω-3 fatty acids reverse the dysregulation of this system, improve insulin sensitivity and control body fat.

Endocannabinoids are lipids, derived from the ω-6 AA. Their concentrations are regulated by a) dietary intake of ω-6 and ω-3 fatty acids; and b) activity of biosynthetic and catabolic enzymes involved in the endocannabinoid pathway, an important factor in regulation of appetite and metabolism. The endocannabinoid system is involved in regulation of energy balance. Sustained hyperactivity of the endocannabinoid system contributes to obesity.

Omega-6 and omega-3 (ω-6 and ω-3) polyunsaturated fatty acids (PUFAs) are essential fatty acids that must be derived from the diet. These special fatty acids cannot be made by humans and other mammals because we lack the enzymes for desaturation. Recent consolidation and standardization of our prepared food sources by agribusiness and large scale food marketers our western diets contain significantly greater levels of ω-6 PUFAs but also significantly diminished levels of ω-3 PUFAs. We now consume an ω-fat diet approximating an unhealthy ω-6/ω-3 ratio of about 20:1, rather than the 1:1 ratio that prevailed during evolution of humans.

The ω-6 PUFAs in our diets are primarily provided by vegetable oils, such as corn oil, while the ω-3 PUFAs are obtained primarily from cold-water fish.

Between these two classes of essential fatty acids (EFA), ω-6 and ω-3, the chemical distinguishing feature between the ω-6 and the ω-3 fatty acids is the location of the first double bond, counting from the methyl end of the fatty acid molecule. ω-6 fatty acids are represented by linoleic acid (LA) (18:2-ω-6) and ω-3 fatty acids by α-linolenic acid (ALA) (18:3-ω-3). LA is plentiful in nature and is found in the seeds of most plants except for coconut, cocoa, and palm. ALA, on the other hand, is found in the chloroplasts of green leafy vegetables, and in the seeds of flax, rape, chia, perilla and walnuts. Both essential fatty acids are metabolized to longer-chain fatty acids of 20 and 22 carbon atoms. LA is metabolized to arachidonic acid (AA) (20:4-ω-6) while ALA is metabolized to eicosapentaenoic acid (EPA) (20:5-ω-3) and docosahexaenoic acid (DHA) (22:6-ω-3). This is achieved by increasing the chain length and the degree of unsaturation by adding extra double bonds to the carboxyl end of the fatty acid molecule. EPA shows more consistent effects in improving mood disorders; while DHA is more often associated with neurodegenerative conditions such as Alzheimer's disease (AD). DHA is quantitatively the most important omega-3 PUFA in the brain. AA is found predominantly in the phospholipids of grain-fed animals, dairy and eggs. EPA and DHA are found in the oils of fish, particularly fatty fish.

DHA [22:6(ω-3)] is preferentially incorporated into brain tissue relative to AA [20:4(ω-6)]; (14, 16-18), a primary source of AEA and AG. The circulatory system delivers AA and DHA to the brain and other tissues either from dietary sources or after synthesis in the liver from shorter-chain essential fatty acids. Adequate intake of DHA and/or eicosapentaenoic acid ([20:5(ω-3)]; EPA) is critical to development of cell membranes, and as such critical to brain development, structure, and function. The brain after water is excluded from the calculation comprises over 50% fatty tissues. The brain structure with its membranous axons and dendrites, heavy concentration of membrane bound organelles and fatty interstitial tissues providing electrical insulation requires these facts for proper function.

Studies show that taking γ-linolenic acid (GLA)—a type of ω-6 fatty acid—for a period of six months or more may reduce symptoms of nerve pain in persons suffering from diabetic neuropathy. The positive effects are more prominent when metabolism is more in balance, such as maintain sugar at proper levels.

Modern agriculture, by changing animal feeds in its emphasis to maximize production, has decreased the ω-3 fatty acid content in many foods: animal meats, eggs, and even fish. Foods from edible wild plants contain a good balance of ω-6 and ω-3 fatty acids. Purslane, a wild plant, in comparison to spinach, red leaf lettuce, buttercrunch lettuce and mustard greens, has eight times more ALA than the cultivated plants. Modern aquaculture produces fish that contain less ω-3 fatty acids than do fish grown naturally in the ocean, rivers and lakes. The fatty acid composition of egg yolk from free-ranging chicken has an ω-6/ω-3 ratio of only 1.3 whereas the United States Department of Agriculture (USDA) egg has a ratio of 19.9.

### Fighting Inflammation

We know inflammation negatively affects our health and can exacerbate and even characterize a disease. Most chronic diseases, including, but not limited to: cancer, diabetes, heart disease, arthritis, AD, etc., have a strong inflammatory component. Because of this, the link between what we eat that may promote or suppress inflammation and disease is critical to our healthy well-beings.

Eating healthy fats, especially PUFAs, generally deliver a positive effect on health. These polyunsaturated fats found in ω-3 and ω-6 fatty acids can play significant role in health and/or disease. GLA is produced in the body from linoleic acid, an ω-6 essential fatty acid. GLA is further metabolized to dihomo-γ-linolenic acid (DGLA), an anti-inflammatory fatty acid. Thus, GLA must be considered to be an-anti-inflammatory nutrient.

A healthy diet will contain a balance of ω-3 and ω-6 fatty acids. ω-3 fatty acids help reduce inflammation, while some ω-6 fatty acids tend to promote inflammation. In fact, some studies suggest that an elevated intake of ω-6 fatty acids may play a role in complex regional pain syndrome. The typical American diet tends to contain 14 to 25 times more ω-6 fatty acids than ω-3 fatty acids.

The Mediterranean diet, on the other hand, has a healthier balance between ω-3 and ω-6 fatty acids. Studies show that people following a Mediterranean-style diet are less likely to develop heart disease. The Mediterranean diet does not include much meat (which is high in ω-6 fatty acids, though grass fed beef has a more favorable ω-3 to ω-6 fatty acid ratio). The Mediterranean diet emphasizes foods richer in ω-3 fatty acids, including whole grains, fresh fruits and vegetables, fish, olive oil, garlic, as well as moderate wine consumption.

A diet with a high ω-6/ω-3 ratio causes an increase in endocannabinoid signaling and related mediators, which lead to an increased inflammatory state, energy homeostasis, and moodiness. Animal experiments reveal that a diet high in ω-6 acid leads to decreased insulin sensitivity in muscle and promotes fat accumulation in adipose tissue. Diets high in ω-3 fatty acids reverse the dysregulation of this system, improve insulin sensitivity and control body fat.

Endocannabinoids are lipids, derived from the ω-6 AA. Their concentrations are regulated by a) dietary intake of ω-6 and ω-3 fatty acids; and b) activity of biosynthetic and catabolic enzymes involved in the endocannabinoid pathway, an important factor in regulation of appetite and metabolism. The endocannabinoid system is involved in regulation of energy balance. Sustained hyperactivity of the endocannabinoid system contributes to obesity.

There are several different types of ω-6 fatty acids, and not all promote inflammation. Most ω-6 fatty acids in the diet come from vegetable oils, such as linoleic acid (LA), not to be confused with α-linolenic acid (ALA), an ω-3 fatty acid. Linoleic acid is converted to γ-linolenic acid (GLA), an ω-6 fatty acid, in the body. GLA can then break down further to arachidonic acid (AA). GLA is found in several plant-based oils, including evening primrose oil (EPO), borage oil, and black currant seed oil.

GLA may actually reduce inflammation. Much of the GLA taken as a supplement is converted to DGLA an ω- an ω-6 fatty acid that fights inflammation. Having enough of certain nutrients in the body (including magnesium, zinc, and vitamins C, B3, and B6) helps to promote conversion of GLA to DGLA.

### ω-6 Fatty Acids

### Arachidonic Acid

Arachidonic acid, can be metabolized by oxygenation into a large family of biologically active substances, the prostanoids. These include the prostaglandins, thromboxanes, prostacyclins, leukotrienes and a selection of related compounds. Oxydation can take place at many positions of arachidonic acid. A cyclo-oxygenase introduces oxygen at C-11 and converts the resulting peroxy compound into a 9, 11-endoperoxide structure. The cyclic peroxides thus formed, PGG2 and PGH2, are highly potent compounds and are the immediate precursors of the prostaglandins, thromboxanes and prostacyclin. Other enzymes, the lipoxygenases, may instead introduce oxygen at C-5, C-8, C-9, C-12 or C-15: further conversions from, for example, the initially formed 5- or 15-hydroperoxy acids may lead to the leukotrienes. The prostanoids display strong and varied biological activities, and have observable effects on numerous processes in the body. In some pathological conditions the prostanoids play important roles. For example, certain products of the arachidonic acid cascade are considered to be mediators of the inflammatory response: they are formed during the process, contribute to the symptoms of erythema, vascular leakage, fever, pain and chemotaxis, and inhibition of their biosynthesis can be achieved at different levels by the anti-inflammatory drugs

The enzymes cyclooxygenase-1 and -2 (i.e.,) prostaglandin G/H synthase 1 and 2 {PTGS1 and PTGS2}) metabolize arachidonic acid to Prostaglandin G2 and prostaglandin H2, which in turn may be converted to various other prostaglandins, to prostacyclin, to thromboxanes, and/or to a 17-carbon product of thromboxane metabolism of prostaglandin G2/H2, 12-hydroxyheptadecatrienoic acid (12-HHT).

The enzyme, 5-lipoxygenase, metabolizes arachidonic acid to 5-hydroperoxyicosa-tetraenoic acid (5-HPETE), which can be metabolized to various leukotrienes including, but not limited to: B4, leukotriene C4, leukotriene D4, and leukotriene E4 as well as to 5-hydroxyicosatetraenoic acid (5-HETE) (which may then be further metabolized to 5-HETE's more potent 5-keto analog, 5-oxo-eicosatetraenoic acid (5-oxo-ETE) and 5-hydroxyicosatetraenoic acid.

The enzymes 15-lipoxygenase-1 (ALOX15) and 15-lipoxygenase-2 (ALOX15B) metabolize arachidonic acid to 15-hydroperoxyicosatetraemoic acid (15-HPETE) which may then be further metabolized to 15-hydroxyicosatetraenoic acid (15-HETE) and lipoxins; 15-lipoxygenase-1 may also further metabolize 15-HPETE to eoxins in a pathway analogous to (and presumably using the same enzymes as used in) the pathway which metabolizes 5-HPETE to leukotrienes.

The enzyme 12-lipoxygenase (ALOX12) metabolizes arachidonic acid to 12-hydroperoxyeicosatetraenoic acid (12-HPETE) which may then be metabolized to 12-hydroxyeicosatetraenoic acid (12-HETE) and to hepoxilins.

Arachidonic acid is reactant in the biosynthesis of anandamide.

Some arachidonic acid is converted into hydroxyeicosatetraenoic acids (HETEs) and epoxyeicosatrienoic acids (EETs) by epoxygenase.

Arachidonic acid promotes the repair and growth of skeletal muscle tissue when converted to prostaglandin PGF2a during and following physical exercise. PGF2a promotes muscle protein synthesis by signaling through the Akt/mTOR pathway, similar to leucine, β-hydroxy β-methylbutyric acid, and phosphatidic acid.

Arachidonic acid is one of the most abundant fatty acids in the brain, and is present in similar quantities to DHA. These two account for approximately 20% of CNS fatty acid content. Unsaturated lipid strings decrease the ability of the fatty acid strands to closely align. Arachidonic acid when incorporated in biological membranes renders increased fluidity. The molecular bends (folds) in the fatty acids thus lowers the membrane melting point. Like DHA, neurological health is reliant upon adequate levels of arachidonic acid. Among other things, arachidonic acid helps to maintain hippocampal cell membrane fluidity. It also helps protect the brain from oxidative stress by activating peroxisome proliferator-activated receptor γ-ARA also activates syntaxin-3 (STX-3), a protein involved in the growth and repair of neurons.

In adults, disturbed metabolism of ARA contributes to neurological disorders such as Alzheimer's disease (AD) and bipolar disorder. Bipolar disorder involves significant alterations in conversion of arachidonic acid to other bioactive molecules (overexpression or disturbances in the ARA enzyme cascade).

Skeletal muscle is an especially active site of arachidonic acid retention, accounting for roughly 10-20% of the phospholipid fatty acid content in muscle on average.

In addition to being involved in cellular signaling as a lipid second messenger involved in the regulation of signaling enzymes, such as PLC-γ, PLC-δ, and PKC-α, -β, and -γ isoforms, arachidonic acid is a key inflammatory intermediate and can also act as a vasodilator.

AA is the precursor of 2-arachidonoylglycerol (2AG) and anandamide (AEA). Increasing the precursor pool of AA increases endocannabinoid signaling (like the munchies associated with pot) to favor weight gain and a metabolic profile associated with obesity. Endocannabinoids activate endogenous cannabinoid CB₁ and CB₂ receptors in the brain, liver, adipose tissue, and the gastrointestinal tract. Activation of CB₁ receptors in the hypothalamus leads to increased appetite and food intake.

Linoleic acid—Not to be confused with linolenic acid, α-linolenic acid, or lipoic acid. Linoleic acid (LA) is a polyunsaturated ω-6 fatty acid. It is a colorless liquid at room temperature. It has a lipid number-name of 18:2 cis,cis-9,12. Linoleic acid is a carboxylic acid with an 18-carbon chain and two cis double bonds; with the first double bond located at the sixth (18-12) carbon from the methyl (omega) end.

LA is a polyunsaturated fatty acid available for the biosynthesis of arachidonic acid (AA) and thus some prostaglandins, leukotrienes (LTA, LTB, LTC), and thromboxane (TXA). It is found in the lipids of cell membranes. It is abundant in many nuts, fatty seeds (flax seeds, hemp seeds, poppy seeds, sesame seeds, etc.) and their derived vegetable oils; comprising over half (by weight) of poppy seed, safflower, sunflower, corn, and soybean oils.

LA is converted by various lipoxygenases, cyclooxygenases, certain cytochrome P450 enzymes (the CYP monooxygenases), and non-enzymatic autooxidation mechanisms to mono-hydroxyl products viz., 13-Hydroxyoctadecadienoic acid and 9-Hydroxyoctadecadienoic acid; both these hydroxy metabolites are enzymatically oxidized to their keto metabolites, 13-oxo-octadecadienoic acid and 9-oxo-octadecdienoic acid. Certain cytochrome P450 enzymes, the CYP epoxygenases, metabolize LA to epoxide products viz., its 12,13-epoxide, Vernolic acid and its 9,10-epoxide, Coronaric acid. All of these LA products have bioactivity and are implicated in human physiology and pathology as indicated in the cited linkages.

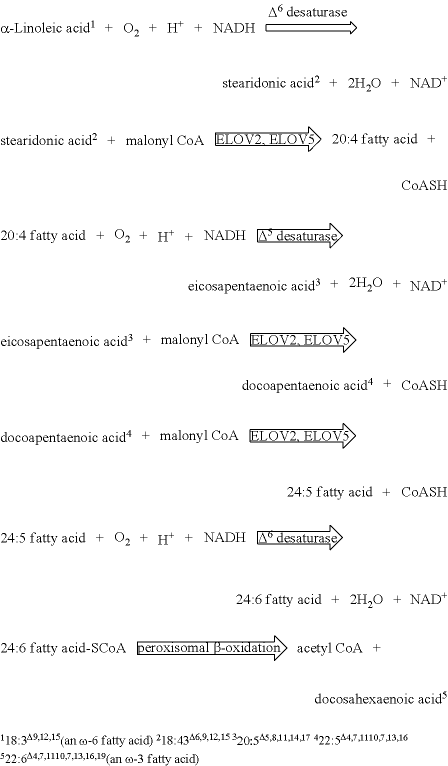

### γ-Linolenic Acid

γ-linolenic acid or GLA (γ-Linolenic acid), (INN and USAN gamolenic acid) is a fatty acid found primarily in vegetable oils. When acting on GLA, 5-lipoxygenase produces no leukotrienes thus in the presence of GLA the conversion by 5-lipoxygenase of arachidonic acid to leukotrienes is inhibited.

### Dihomo-γ-Linolenic Acid

Dihomo-γ-linolenic acid (DGLA) is a 20-carbon ω-6 fatty acid. It goes by the name 20:3 (ω-6). DGLA is a carboxylic acid with a 20-carbon chain and three cis double bonds; the first double bond is located at the sixth carbon from the ω end. DGLA is the elongation product of γ-linolenic acid (GLA; 18:3, ω-6). GLA, in turn, is a desaturation product (Δ6 desaturase) of linoleic acid (18:2, ω-6). DGLA is made in the body by the elongation of GLA, by a very efficient enzyme that does not appear to suffer any form of (dietary) inhibition. DGLA is an extremely uncommon fatty acid, found only in trace amounts in animal products. DGLA production from GLA is enhanced when high levels of α-linolenic acid are present, blocking the arachidonic acid pathway.

The eicosanoid metabolites of DGLA include:

Series-1 thromboxanes (thromboxanes with 1 double-bond), via the COX-1 and COX-2 pathways.

Series-1 prostanoids, via the COX-1 and COX-2 pathways.

A 15-hydroxyl derivative that blocks the transformation of arachidonic acid to leukotrienes.

All of these effects are anti-inflammatory. This is in marked contrast with the analogous metabolites of arachidonic acid (AA), which are the series-2 thromboxanes and prostanoids and the series-4 leukotrienes. In addition to yielding anti-inflammatory eicosanoids, DGLA competes with AA for COX and lipoxygenase, inhibiting the production of AA's eicosanoids.

### Docosatetraenoic Acid

All-cis-7,10,13,16-docosatetraenoic acid is an ω-6 fatty acid 22 carbons in length with the trivial name adrenic acid (AdA). This is a naturally occurring polyunsaturated fatty acid formed through a 2-carbon chain elongation of arachidonic acid. It is one of the most abundant fatty acids in the early human brain. This unsaturated fatty acid is also metabolized by cells to biologically active products viz., dihomoprostaglandins, dihomo-epoxyeicosatrienoic acids, and dihomo-EETs.

### Eicosatetraenoic Acid

Eicosatetraenoic acid (ETA) designates any straight chain 20:4 fatty acid.

ETA has two isomers, one ω-6 and 1 ω-3, both of them also essential fatty acids.

All-cis 5,8,11,14-eicosatetraenoic acid is in fact an ω-6 fatty acid with the trivial name arachidonic acid. It is formed by a desaturation of dihomo-γ-linolenic acid (DGLA, 20:3 ω-6).

All-cis 8,11,14,17-eicosatetraenoic acid is an ω-3 fatty acid. It is an intermediate between stearidonic acid (18:4 ω-3) and eicosapentaenoic acid (EPA, 20:5 ω-3).

ω-3 DPA is an ω-3 fatty acid with the trivial name clupanodonic acid. It is an intermediary between eicosapentaenoic acid (EPA, 5,8,11,14,17-20:5n-3) and docosahexaenoic acid (DHA, 22:6 ω-3) in the following stepwise pathway in which DPAn-3 is a precursor to DHA (6,9,12,15,18,21:24:6n-3) and the final product, 4,7,10,13,16,19-22:6n-3, can be retro-converted to DPAn-3.

Mammalian cells, including human cells, metabolize DPAn-3 to an array of products that are members of the specialized proresolving mediators class of PUFA metabolites. These metabolites include seven resolvins Ds (RvT1, RvT2, RvT3, RvT4, RvD1n-3, RvD2n-3, and RvD5n-3; see specialized proresolving mediators#n-3 DPA-derived resolvins and Resolvin); two protectins (PD1n-3 and PD2n-3; n-3 DPA-derived protectins/neuroprotectins and neuroprotectin); and three maresins (MaR1n-3, MaR2 n-3,and MaR3n-3l.

### ω-3 Fatty Acids

Stimulation of GPR120 by ω-3 PUFAs has been shown to result in elevation of intracellular Ca2+ levels and activation of the extracellular signal-regulated kinase (ERK) family cascade through the associated Gq- and G11-type G-proteins.

GPR120 (FFAR4) is highly expressed in adipose tissue, and proinflammatory macrophages. The high expression level of GPR120 in mature adipocytes and macrophages is indicative of the fact that GPR120 is likely to play an important role in biologic functions of these cell types. In contrast, negligible expression of GPR120 is seen in muscle, pancreatic (3-cells, and hepatocytes. Although not expressed at appreciable levels in hepatocytes expression of GPR120 is highly inducible in liver resident macrophage-like cells known as Kupffer cells. GPR120 can be activated with a synthetic agonist (GW9508) as well as the ω-3 PUFAs, EPA and DHA. GPR120 is also expressed in enteroendocrine L cells of the gut. These are the cell types that express the incretin peptide hormone GLP-1. Previous work on GPR120 focused on the potential ability of this receptor to stimulate L cell GLP-1 secretion.

ω-3 fatty acids are found in fatty layers of cold-water fish and shellfish, plant and nut oils, English walnuts, flaxseed, algae oils, and fortified foods. You can also get ω-3s as supplements. Food and supplement sources of these fatty acids differ in the forms and amounts they contain.

Hundreds of studies suggest that ω-3s may provide some benefits to a wide range of diseases: cancer, asthma, depression, cardiovascular disease, ADHD, and autoimmune diseases, such as rheumatoid arthritis.

How could fatty acids be so beneficial for so many different conditions?

“All these diseases have a common genesis in inflammation,” says Joseph C. Maroon, MD, professor and vice chairman of the department of neurological surgery at the University of Pittsburgh School of Medicine. Co-author of Fish Oil: The Natural Anti-Inflammatory, Maroon says that in large enough amounts, ω-3's reduce the inflammatory process that leads to many chronic conditions.

For these and other reasons, the Department of Health and Human Services (HHS), the U.S. Department of Agriculture (USDA), the American Heart Association, and the American Dietetic Association recommend eating two 8-ounce servings of fish each week.

There are the two main types of ω-3 fatty acids:

Long-chain ω-3 fatty acids are EPA and DHA. These are plentiful in fish and shellfish. Algae often provide only DHA.

Short-chain ω-3 fatty acids are ALA (α-linolenic acid). These are found in plants, such as flaxseed. Though beneficial, ALA ω-3 fatty acids have less potent health benefits than EPA and DHA. You'd have to eat a lot to gain the same benefits as you do from fish.

Children require DHA for growth and development, and the brain, CNS and retina rely heavily on the adequate supply of DHA during growth in the womb. Thus women should emphasize DHA in their diets when they become pregnant and continue to take this until they cease breastfeeding. Children continue to need DHA up until the age they start school, so if children under the age of five are taking an ω-3 supplement, it should contain DHA. The exception is for children with developmental problems—where pure EPA or high EPA ω-3 has been shown to be most effective for supporting cognitive function.

Between the ages of five and 65, the majority of the body's needs can be met by using EPA-rich oils and eating fish, marine products, organic greens and pastured animal products. EPA levels are under constant demand and low EPA levels in adolescents and adults correlates strongly with development of mental health issues, including depression, dyslexia and dyspraxia, heart problems, joint and bone conditions, as well as neurodegenerative diseases such as MS and Parkinson's. EPA also protects our genes and cell cycle, as well as helping to keep our stress response regulated, so an adequate supply of EPA throughout adult life can help prevent a range of chronic illness.

ω-3 (n-3)] long-chain PUFA, including EPA and DHA, are dietary fats with an array of health benefits. They are incorporated in many parts of the body including cell membranes and play a role in anti-inflammatory processes and in the viscosity of cell membranes. EPA and DHA are essential for proper fetal development and healthy aging. DHA is a key component of all cell membranes and is found in abundance in the brain and retina. EPA and DHA are also the precursors of several metabolites that are potent lipid mediators, considered by many investigators to be beneficial in the prevention or treatment of several diseases.

In later life, cognitive function and brain deterioration may become a concern. Once again, maintaining high levels of EPA has been shown to lower the risk of developing and worsening cognitive decline and dementia. If, however, you know someone who already has a diagnosis of dementia or Alzheimer's (AD), their brain has already been damaged and needs structural support. At this point, DHA becomes important again and taking a high-EPA product that contains 250 mg of DHA also is important to prevent further loss of brain tissue.

Cardiovascular disease is the cause of 38% of all deaths in the United States, many of which are preventable. Chronic inflammation is thought to be the cause of many chronic diseases, including cardiovascular disease. EPA and DHA are thought to have anti-inflammatory effects and a role in oxidative stress and to improve cellular function through changes in gene expression. In a study that used human blood samples, EPA+DHA intake changed the expression of 1040 genes and resulted in a decreased expression of genes involved in inflammatory and atherogenesis-related pathways, such as nuclear transcription factor κB signaling, eicosanoid synthesis, scavenger receptor activity, adipogenesis, and hypoxia signaling. Circulating markers of inflammation, such as C-reactive protein (CRP), TNF α, and some ILs (IL-6, IL-1), correlate with an increased probability of experiencing a cardiovascular event. Inflammatory markers such as IL-6 trigger CRP to be synthesized by the liver, and elevated levels of CRP are associated with an increased risk of the development of cardiovascular disease. A study of 89 patients showed that those treated with EPA+DHA had a significant reduction in high-sensitivity CRP (66.7%, P<0.01) (33). The same study also showed a significant reduction in heat shock protein 27 antibody titers (57.69%, P<0.05), which have been shown to be overexpressed in heart muscle cells after a return of blood flow after a period of ischemia (ischemia-reperfusion injury) and may potentially have a cardioprotective effect.

ω-3's downregulate enzymes or pathways including, but not limited to: the MAPKKK cascade, IL-(1-7, 8), adipogenesis, fatty acid ω oxidation, lipid transport, glycogen metabolism, Krebs cycle, transcription cycle binding, peptide GPCRs, EGFR1, etc. and upregulate enzymes or pathways including, but not limited to: translation initiation factor, DNA replication reactome, RNA transcription reactome, cell cycle KEGG, cytokinesis, etc. EPA and DHA are the dynamic duo of bio-available ω 3s. EPA and DHA may have increased benefits when consumed together. Benefits include:
slowing progression of age-related memory loss
supporting memory and learning ability including focus and attention
supporting healthy brain function and promote a positive mood and well-being
Other benefits of fish oil containing both EPA and DHA are that they may help to maintain healthy triglyceride levels, while promoting the metabolism of dietary fat and cholesterol.

ω-3 fatty acids—especially EPA and DHA—have been shown to reduce inflammation and may help prevent chronic diseases, such as heart disease and arthritis. They may also be important for brain health and development, as well as normal growth and development.

### α-Linolenic Acid

α-linolenic acid is a kind of ω-3 fatty acid found in plants. It is found in flaxseed oil, and in canola, soy, perilla, and walnut oils.

α-linolenic acid is similar to the EPA and DHA ω-3 fatty acids that are in fish oil. α-linolenic acid is convertible into EPA and DHA, but this is not a highly used pathway. eicosapentaenoic acid

EPA is a polyunsaturated fatty acid (PUFA) that acts as a precursor for prostaglandin-3 (which inhibits platelet aggregation), thromboxane-3, and leukotriene-5 eicosanoids. Studies of fish oil supplements, which contain EPA, have failed to support claims of preventing heart attacks or strokes.

Intake of large doses (2.0 to 4.0 g/day) of long-chain ω-3 fatty acids as prescription drugs or dietary supplements are generally required to achieve significant (>15%) lowering of triglycerides, and at those doses the effects can be significant (from 20% to 35% and even up to 45% in individuals with levels greater that 500 mg/dL).

### Docosahexaenoic Acid

Docosahexaenoic acid (DHA) is an ω-3 fatty acid that is a primary structural component of the human brain, cerebral cortex, skin, and retina. It can be synthesized from α-linolenic acid or obtained directly from maternal milk (breast milk), fish oil, or algae oil. DHA enhances LKB1 signaling and acts as a cannabinoid when it binds and activates PPARα.

Most of the DHA in fish and multi-cellular organisms with access to cold-water oceanic foods originates from photosynthetic and heterotrophic microalgae, and becomes increasingly concentrated in organisms the further they are up the food chain.

DHA comprises 40% of the polyunsaturated fatty acids (PUFAs) in the brain and 60% of the PUFAs in the retina. Fifty percent of the weight of a neuron's plasma membrane is composed of DHA. DHA modulates the carrier-mediated transport of choline, glycine, and taurine, the function of delayed rectifier potassium channels, and the response of rhodopsin contained in the synaptic vesicles, among many other functions.

DHA can be metabolized into DHA-derived specialized pro-resolving mediators (SPMs), DHA epoxides, electrophilic oxo-derivatives (EFOX) of DHA, neuroprostanes, ethanolamines, acylglycerols, docosahexaenoyl amides of amino acids or neurotransmitters, and branched DHA esters of hydroxy fatty acids, neuroprostanes (e.g., A4-, D4-, E4-, F4-, and J4-neuroprostanes), synaptamide (an ω-3 analogue of AEA), epoxydocosapentaenoic acids, etc.

The enzyme CYP2C9 metabolizes DHA to epoxydocosapentaenoic acids (EDPs; primarily 19,20-epoxy-eicosapentaenoic acid isomers [i.e. 10,11-EDPs]). These may act similar to cannabinoids with anti-hypertensive, anti-arrhythmic, anti-VEGF, anti-FGF2, and anti-mitotic effects.

### Stearidonic Acid

Stearidonic acid (SDA) is an ω-3 fatty acid, sometimes called moroctic acid. It is biosynthesized from α-linolenic acid by the enzyme delta-6-desaturase. Natural sources of this fatty acid are the seed oils of hemp, blackcurrant, corn gromwell and echium (although the plant is a source of stearidonic acid, it is toxic for human consumption), and the cyanobacterium Spirulina.

### Eicosatetraenoic Acid

Eicosatetraenoic acid (ETA) designates any straight chain 20:4 fatty acid. All-cis 8,11,14,17-eicosatetraenoic acid is an ω-3 fatty acid. It is an intermediate between stearidonic acid (18:4 ω-3) and eicosapentaenoic acid (EPA, 20:5 ω-3)

ETA has two isomers one that is ω-3 and one that is ω-6, both of them also essential fatty acids, that are of particular interest for that reason.

All-cis 5,8,11,14-eicosatetraenoic acid is in fact an ω-6 fatty acid with the trivial name arachidonic acid. It is formed by a desaturation of dihomo-γ-linolenic acid (DGLA, 20:3 ω-6).

### Eicosapentaenoic Acid (EPA)

EPA is an ω-3 fatty acid. In physiological literature, it is given the name 20:5(n-3). It also has the trivial name timnodonic acid. In chemical structure, EPA is a carboxylic acid with a 20-carbon chain and five cis double bonds; the first double bond is located at the third carbon from the ω end.

EPA is a polyunsaturated fatty acid (PUFA) that acts as a precursor for prostaglandin-3 (which inhibits platelet aggregation), thromboxane-3, and leukotriene-5 eicosanoids. It is obtained in the human diet by eating oily fish or fish oil, e.g. cod liver, herring, mackerel, salmon, menhaden and sardine, and various types of edible seaweed and phytoplankton. It is also found in human breast milk.

The human body converts α-linolenic acid (ALA) to EPA. ALA is itself an essential fatty acid, an appropriate supply of which must be ensured. The efficiency of the conversion of ALA to EPA, however, is much lower than the absorption of EPA from food containing it. Because EPA is also a precursor to DHA, ensuring a sufficient level of EPA on a diet containing neither EPA nor DHA is harder both because of the extra metabolic work required to synthesize EPA and because of the use of EPA to metabolize into DHA. Medical conditions like diabetes or certain allergies may significantly limit the human body's capacity for metabolization of EPA from ALA.

### Docosapentaenoic Acid

Docosapentaenoic acid (DPA) designates any straight chain 22:5 fatty acid, that is a straight chain open chain type of polyunsaturated fatty acid (PUFA) which contains 22 carbons and 5 double bonds. DPA (ω-3) is primarily used to designate two isomers, all-cis-4,7,10,13,16-docosapentaenoic acid (i.e. 4Z,7Z,10Z,13Z,16Z-docosapentaenoic acid) and all-cis-7,10,13,16,19-docosapentaenoic acid (ω-6) (i.e. 7Z,10Z,13Z,16Z,19Z-docosapentaenoic acid). ω-3 DPA is an ω-3 fatty acid with the trivial name clupanodonic acid. It is an intermediary between eicosapentaenoic acid (EPA, 5,8,11,14,17-20:5n-3) and docosahexaenoic acid (DHA, 22:6 ω-3) in the following stepwise pathway in which DPAn-3 is a precursor to DHA (6,9,12,15,18,21:24:6n-3) and the final product, 4,7,10,13,16,19-22:6n-3, can be retro-converted to DPAn-3.

Mammalian cells, including human cells, metabolize DPAn-3 to an array of products that are members of the specialized proresolving mediators class of PUFA metabolites. These metabolites include seven resolvins Ds (RvT1, RvT2, RvT3, RvT4, RvD1n-3, RvD2n-3, and RvD5n-3; see specialized proresolving mediators n-3 DPA-derived resolvins and Resolvin); two protectins (PD1n-3 and PD2n-3; and three maresins (MaR1n-3, MaR2n-3, and MaR3n-3l). Clupanodonic acid, along with its metabolite DHA and other long chain ω-3 fatty acids, is under study to determine properties of ω-3 fats in humans, such as in inflammation mechanisms.

ω-6 DPA is an ω-6 fatty acid with the trivial name osbond acid. It is formed by the stepwise elongation and desaturation of arachidonic acid i.e. 5Z,8Z,11Z,14Z-eicosatetraenoic acid (5,8,11,14-20:4n-6) to the 24 carbon PUFA intermediate (i.e. 6,9,12,15,18:24-5 n-6) and the retro-conversion of this intermediate to DPA n-6 as: all-cis-7,10,13,16,19-docosapentaenoic acid (clupanodonic acid).

They are also commonly termed ω-3 DPA and ω-6 DPA, respectively; these designations describe the position of the double bond being 6 or 3 carbons closest to the (ω) carbon at the methyl end of the molecule and is based on the biologically important difference that ω-6 and ω-3 PUFA are separate PUFA classes, i.e. the ω-6 fatty acids and ω-3 fatty acids, respectively. Mammals, including humans, cannot interconvert these two classes and therefore must obtain dietary essential PUFA fatty acids from both classes in order to maintain normal health.

### Tetracosahexaenoic Acid, aka Nisinic Acid

Nisinic acid is a very long chain polyunsaturated ω-3 fatty acid, much like DHA. The lipid name is 24:6 (n-3) and the chemical name is all-cis-6,9,12,15,18,21-tetracosahexaenoic acid. It is not well studied as there are hurdles to studies, but PUFA molecules even longer than DHA, nisinic acid included, should be evaluated as positive supplements.

It appears that both EPA and DHA lower triglycerides, however DHA appears to raise low-density lipoprotein (the variant which drives atherosclerosis; sometimes inaccurately called: “bad cholesterol”) and LDL-C values (always only a calculated estimate; not measured by labs from person's blood sample for technical and cost reasons), while EPA does not.

Most of the DHA in fish and multi-cellular organisms with access to cold-water oceanic foods originates from photosynthetic and heterotrophic microalgae, and becomes increasingly concentrated in organisms the further they are up the food chain.

The big difference in effect between dietary supplement and prescription forms of ω-3 fatty acids is that the prescription variants are concentrated to markedly increase the amount of these key fatty acids per capsule over the many other fats present in “fish oil” and the mercury, also present in “fish oil”, has been removed.

ω-3 fatty acids can be damaging to mitochondria. Mitochondria robustly produce reactive oxygen species (ROS). Unmanaged production of ROS causes oxidative damage to biomacromolecules, especially including lipid peroxidation, damaged lipids adversely impact cellular membrane structure and functions. Mitochondria, the main target of ROS-induced damage, are equipped with a network of antioxidants that control ROS production. Dietary intake of ω-3 polyunsaturated fatty acids (ω3PUFAs) and consequently any increase in ω3PUFA content of membrane lipids may be disadvantageous to the health under some circumstances because ROS-induced oxidative peroxidation of ω3PUFAs within membrane phospholipids can lead to the formation of toxic products. Mitochondrial control of lipid peroxidation is one of the mechanisms that protect cell against oxidative damage.

In the past three decades, total fat and saturated fat intake as a percentage of total calories has continuously decreased in Western diets, while the intake of ω-6 fatty acid increased and the ω-3 fatty acid decreased, resulting in a large increase in the ω-6/ω-3 ratio from 1:1 during evolution to 20:1 today or even higher. This change in the composition of fatty acids parallels a significant increase in the prevalence of overweight and obesity. Experimental studies have suggested that ω-6 and ω-3 fatty acids elicit divergent effects on body fat gain through mechanisms of adipogenesis, browning of adipose tissue, lipid homeostasis, brain-gut-adipose tissue axis, and most importantly systemic inflammation. The ω-6 arachidonic acid works through the enzymes cyclooxygenase-1 and -2, aka: prostaglandin G/H synthase 1 and 2, to metabolize arachidonic acid to prostaglandin G2 and prostaglandin H2. These are source material for conversion to several more prostaglandins, prostacyclins, thromboxanes, and 12-hydroxyheptadecatrienoic acid (12-HHT).

As discussed above, AA is processed by 5-lipoxygenase to 5-hydroperoxyicosatetraenoic acid (5-HPETE), to make several leukotrienes and 5-hydroxyicosatetraenoic acid (5-HETE) a source of 5-oxo-eicosatetraenoic acid (5-oxo-ETE). Several additional active compounds including, but not limited to: 15-hydroperoxyicosatetraemoic acid (15-HPETE), 15-hydroxy-icosatetraenoic acid (15-HETE), lipoxins, eoxins, 12-hydroperoxyeicosatetraenoic acid (12-HPETE), 12-hydroxyeicosatetraenoic acid (12-HETE), hepoxilins, anandamide, etc. The AA ω-fatty acid is subject to multiple controls and metabolic switches.

Prospective studies clearly show an increase in the risk of obesity as the level of ω-6 fatty acids and the ω-6/ω-3 ratio increase in red blood cell (RBC) membrane phospholipids, whereas high ω-3 RBC membrane phospholipids decrease the risk of obesity. Recent studies in humans show that in addition to absolute amounts of ω-6 and ω-3 fatty acid intake, the ω-6/ω-3 ratio plays an important role in increasing the development of obesity via both AA eicosanoid metabolites and hyperactivity of the cannabinoid system, which can be reversed with increased intake of EPA and DHA. A balanced ω-6/ω-3 ratio is important for health and in the prevention and management of obesity.

Eicosanoid products derived from ω-6 PUFAs (such as prostaglandin (PG) E2 and leukotriene (LT) B4 synthesized from arachidonic acid (AA)) are more potent mediators of thrombosis and inflammation than similar products derived from ω-3 PUFAs (PGE3 and LTB5 synthesized from EPA. An unbalanced ω-6/ω-3 ratio in favor of ω-6 PUFAs is highly prothrombotic and proinflammatory, which contributes to the prevalence of atherosclerosis, obesity, and diabetes.

5-HT_{1A} receptor agonists are neuromodulators. They decrease blood pressure and heart rate by inducing peripheral vasodilation and by stimulating the vagus nerve. These central mediated effects are the result of activation of 5-HT_{1A} receptors within the rostral ventrolateral medulla. This effect can spread to the periphery to dilate blood vessels in the skin and thereby reduce body temperature. 5-HT_{1A} receptors in other locations are less well characterized, but are under active investigation.

### PPARs

Peroxisome proliferator-activated receptors (PPARs) are a group of nuclear receptor proteins that function as transcription factors regulating the expression of genes. PPARs play essential roles in the regulation of cellular differentiation, development, and metabolism (carbohydrate, lipid, protein) and tumorigenesis. PPARs heterodimerize with the retinoid X receptor (RXR) to bind to specific regions on the DNA of target genes. The RXR also forms a heterodimer with other factors such as vitamin D and thyroid hormone.

PPARδ is a nuclear hormone receptor with apparent involvement in several chronic diseases, including diabetes, obesity, atherosclerosis, and cancer. PPARs can be activated endogenously by signaling metabolites including, but not limited to: unsaturated fatty acids linolenic acid, linoleic acid, petroselenic acid, arachidonic acid, etc.

AEA, 2AG, noladin ether, O-arachidonoyl ethanolamine (virodhamine, O-AEA), oleoylethanolamide (OEA) and palmitoylethanolamide (PEA) activate PPARα. AEA and 2AG activate PPARγ. And like in the example of 2AG and COX2, some PPARγ effects may derive from binding metabolites of the endocannabinoids rather than the endocannabinoid.

PPARγ activity is most profound in fat and glucose metabolism with high levels of expression in the colon and adipose tissues. Macrophages are another high expressing cell. In addition to the endocannabinoids many polyunsaturated fats also interact with PPARγ.

In addition to contributing to proliferation of peroxisomes, PPARα is a transcription factor involved in lipid metabolism, especially in the liver. Energy deprivation elicits a cascade that includes activating PPARα which is essential for ketogenesis. Activating PPARα promotes uptake, utilization, and catabolism of fatty acids by upregulation of genes involved in fatty acid transport, fatty acid binding and activation, and peroxisomal and mitochondrial fatty acid β-oxidation. Expression is especially robust in liver, kidney and heart with its target enzymes relating to the above activities.

PPARα is active in body weight control. Activation with an endocannabinoid like OEA or a synthetic or natural analogue reduces serum cholesterol and in animals, ad libitum food intake. Oxytocin, the hormone encouraging delivery, breast feeding, social interactions and general satisfaction is upregulated in response to OEA. AEA has similar effects but often in different tissues.

PPARδ activates transcription of a variety of target genes by binding to specific DNA elements. Well-described target genes of PPARδ include PDK4, ANGPTL4, PLIN2, and CD36. The expression of this gene is found to be elevated in colorectal cancer cells. The elevated expression can be repressed by adenomatosis polyposis coli (APC), a tumor suppressor protein in the APC/β-catenin signaling pathway.

### GPRs

GPR55, a mammalian G-protein expressed in areas including, but not limited to: cerebral cortex, appendix, lymph nodes, tonsils, spleen, lung, gall bladder, GI tract tissues (e.g., esophagus, salivary glands, small intestine, duodenum, rectum, colon, stomach), testis, breast, skin, etc. is activated by the plant cannabinoids THC and TBD. AEA, 2AG and noladin ether act as endocannabinoids with respect to this receptor. Synthetic cannabinoid CP-55940 agonizes GPR55 and 5-HT_{2A}. Lysophosphatidylinositol and 2-arachidonoyl lysophosphatidylinositol also bind GPR55. Lysophosphatidylinositol has activity on GPR55 similar to analgesic activity cannabinoids have on CB₁. GPRs are active in many biologic and neurologic events including, but not limited to: addiction, anxiety, appetite, nausea, pain, sleep, vomiting.

Endocannabinoids are critical participants in central and peripheral regulation of appetite and body weight. Cannabinoid activation increases desire for and consumption of excess rich, non-nutritious foodstuffs, while antagonizing these receptors decreases self-feeding. The cannabinoid system influences energy storage into fat, glucose homeostasis and insulin sensitivity. GPR55 is one receptor protein known to play an important role in energy homeostasis. Higher levels of GPR55 expression in visceral fat correlates with higher weight and percentage body fat. Higher levels of GPR55 mRNA are found in visceral and subcutaneous fat cells themselves.

GPR55 receptors are expressed in human bone cells and are involved in regulating bone mass. GPR55 is expressed in both osteoblasts and osteoclasts. GPR55 activation induces osteoclastogenesis, cell polarization and bone resorption, but its effect on anabolism is not understood. GPR55 signaling in osteoclasts leads to activation of RhoA and ERK1/2, and effects on the actin cytoskeleton.

Agonizing GPR55 has tendencies to increase weight gain and fat storage including food intake while knocking down GPR55 has opposite effects.

AEA is released by vascular endothelial cells and can bind both CB1 and GPR55 on the surface of the endothelium. Both CB₁ antagonists and GPR55 antagonists inhibit increased Ca²⁺ normally induced by AEA.

In endothelial cells, CB₁ and GPR55 are active, are both are cannabinoid-sensitive with their downstream signaling interacting to influence the other's outcome. E.g., when integrins are unclustered, anandamide activates the CB₁-Gαi-Syk pathway, which inhibits the GPR55-PI3K-Bmx-PLC-Ca²⁺ cascade at the level of Syk. However, when integrins cluster CB₁ uncouples from β1-integrin and releases an uninhibited GPR55-PI3K-Bmx-PLC-Ca²⁺ cascade resulting from anandamide stimulation. Cannabinoids can induce diverse responses in blood cells, such as migration, proliferation, cytokine production, apoptosis, ROS production and chemotaxis, with certain cannabinoid ligands involved in therapeutic immunosuppressant activities. GPR55 expression is significant in many of these cell types with LPI and AM251 agonizing GPR55 in human neutrophils to promote RhoA-dependent chemotaxis. When co-activated, GPR55 and CB₂ signaling pathways exert synergistic potentiation of the RhoA mediated chemotaxis. In the vasculature and blood, GPR55 may be expressed on the same cells and activated by the same cannabinoid ligands to induce both GPR55-derived effects and effects on downstream signaling of the traditional cannabinoid receptors. Cannabidiol (CBD) is a plant derived cannabinoid. Though chemically very similar THC and thus classifiable chemically a cannabinoid for that reason alone. CBD, exerts its physiologic effects through mechanisms distinct from the psycho-active THC. CBD exerts an antidepressant effect by binding the G-coupled protein receptor, hydroxytryptamine serotonin receptor (HTSR). The HTSR is another member of the GPR family. Another plant cannabinoid, cannabdiolic acid (CBDA) has an even stronger affinity for HTSR. CBD or CBDA binding inhibits HTSR signaling so that stronger serotonin or analogue excitatory neurotransmitter signals are necessary. CBD has a profound antagonistic effect on GPR₅₅, regarding its bone density and blood pressure regulation. CBD is also active with receptors in the cerebellum, jejunum and ileum.

Coupled with G-protein G₁₃, GPR₅₅ participates in pathways that stimulate rhoA (active in cytoskeleton and intracellular transport, cell cycling, transcription, tissue polarization, and mitosis), cdc42 (a Rho family GTPase active in cell cycling, endocytosis, cytoskeletal activity, and cell adhesion), and rac1 (the premier member of the Rac subfamily of the Rho family). Rac1 is involved in cell adhesion, differentiation, cell cycling, transport of GLUT4 vesicles to the plasma membrane for insulin dependent glucose uptake. Results of rac1 activation contribute to forming symptoms of type 2 diabetes.

CBD is a receptor stimulator for GABA receptors, rendering GABA more influential in its calming effect, but a repressor for CB₁, interestingly, lowering the receptor's affinity for THC thereby rendering THC less efficacious.

CBD also exerts direct influence on the TRPV₁ receptor/ion channel, a channel active in pain sensation and sensitive to capsaicin (pepper) H⁺ ion, and anandamide, an endogenous cannabinoid.

GPR119 is expressed predominantly in the pancreas and gastrointestinal tract. GPR119 is often co-expressed with pancreatic polypeptide (PPY). GPR119 is a Gs-coupled receptor that upon activation increases intracellular cAMP. Oleamide is at least one cannabinoid that activates GPR119. Classically non-cannabinoid lipid elements, oleoyl-lysophosphatidylcholine (18:1-LPC) and palmitoyl-lysophosphatidylcholine (16:0-LPC) may indirectly or directly activate GPR119.

### Ion Channel as Receptor

Transient receptor potential vanilloid type 1 (TRPV₁) is another cannabinoid receptor found in multiple membranes, including mitochondrial membranes. TRPV is a ligand-gated nonselective ion channel whose expression levels can be controlled with salt management. At least the endocannabinoid AEA activates TRPV₁. This receptor may be coopted for controlling intracellular and extracellular fluid volumes, including organelle volumes and physiologic outcomes such as blood pressure, lymph flow, renal function and cardiac output.

In 2012, French scientists reported the presence of cannabinoid receptors on the membranes of mitochondria. Defects in mitochondria have been linked a wide range of neurodegenerative, autoimmune and metabolic disorders—Alzheimer's, schizophrenia, autism, cancer, epilepsy, diabetes, cardiovascular and neuromuscular disease, and more. Endocannabinoids acting through receptors in mitochondrial membranes can be commandeered to help manage these diseases.

And apart from their action on receptors several endocannabinoids act as antioxidants able to protect cells and their mitochondria from effects of mitochondrial released ROS.

By effectively neutralizing free radicals and mitigating oxidative stress, antioxidants confer a broad range of therapeutic benefits—from slowing down the aging process to reducing the risk of DNA damage linked to cancer. THC and CBD are both potent antioxidants as seen in U.S. Pat. No. 7,094,794.

Oxidative damage can be repaired by removing the damaged organelle through the process of autophagy, whereby faulty cell parts—misfolded or aggregated proteins, dysfunctional mitochondria, etc.—are catabolized and replaced by newly produced components.

When activated, CB₁ receptors present in outer mitochondrial membranes decrease cAMP concentration, protein kinase A activity, complex I activity, and mitochondrial respiration. In the mitochondrial membrane, activating CB₁ regulates energy metabolism.

CB₁ agonists, THC, AEA and HU-210, decrease O₂ consumption at the mitochondrion. Specific enzymatic groups show their susceptibility to control by endocannabinoids. Complex 1 activity rapidly and transiently increases (25% and 50%, respectively for HU210 and THC) with a sudden decrease at a threshold around 10 μM to a loss of 75% or greater activity at >20-40 μM. AEA showed a steady logarithmic decline from about 5-200 μM. Complex 2/3 inhibition is affected differently, AEA and HU210 each elicit a rapid decrease after threshold (2-4 μM), while THC has a more steady logarithmic concentration dependent effect. AEA had no effect on H₂O₂ production while both THC and HU210 cause a H₂O₂ increase between 2 and 10 μM.

According to a 2016 report in Philosophical Transactions of the Royal Society (London): “Cannabinoids as regulators of mitochondrial activity, as anti-oxidants and as modulators of clearance processes protect neurons on the molecular level . . . Neuroinflammatory processes contributing to the progression of normal brain ageing and to the pathogenesis of neurodegenerative diseases are suppressed by cannabinoids, suggesting that they may also influence the aging process on the system level.”

Given the many diseases associated with age that include major mitochondrial metabolic components, In addition to mitochondrial manipulation by supplementing with compounds including, but not limited to: mitochondrial electron transport chain enhancer, dichloroacetic acid, inhibitor of lactate production, compound modulating amino acid availability, compound modulating glucose availability, palmitic acid, ketogenesis inhibitor, PIP₂ pathway modulator, an Fe—S complex disruptor, dehydroascorbic acid, ascorbic acid mTORC1 modulator, B₁₂, a ubiquitination stimulant, a ubiquitination inhibitor, a deubiquitination stimulant, a deubiquitination inhibitor, oxidoreductase stimulator, glutamate dehydrogenase stimulator, aspartate transaminase inhibitor, caveolin 1 modulator, a flavone, a flavonoid, glucose-6-phosphate dehydrogenase inhibitor, 6-phosphogluconolactonase inhibitor, pyruvate dehydrogenase inhibitor, α-ketoglutarate dehydrogenase inhibitor, vitamin K, lactate dehydrogenase inhibitor, moncarboxylate transport inhibitor, staurosporine, omega 3, 6-phosphogluconate dehydrogenase inhibitor, NFkB inhibitor, melatonin, α-ketoglutarate, dichloroacetate, B3, B5, D2 and analogues thereof, an inhibitor of an iron-sulfur protein, D3 and analogues thereof, leucine, isoleucine, valine, GDP, L-carnitine, acetyl-L-carnitine, vitamin B5, resveratrol, CoQ10, α-lipoic acid, selenium, nicotinamide, adenine dinucleotide enhancing supplement, vitamin B3, GTP, L-alanine, melatonin, etc., cannabinoid compounds and there antagonists could beneficially be incorporated with or separately provided for similar effect.

1. A method of advancing physiological welfare in a mammal, said method comprising: in an individual identified as being compromised by at least one cannabinoid related physiological deficit, delivering to said individual a combination of cannabinoid activity and/or cannabinoid effect modulating interventions selected to ameliorate at least one of said cannabinoid related physiological deficits with an effect that at least one of said cannabinoid related physiological deficits is ameliorated.

2. The method of claim 1 wherein the modulated cannabinoid activity and/or cannabinoid effect comprises binding at least one selected from the group consisting of: 5-HT_{1A}, 5-HT_{2A}, CB₁, CB₂, TRPV₁, GPR18, GPR55, GPR119, GPR120, PPARα, PPARγ and PPARδ.

3. The method of claim 1 wherein the modulated cannabinoid activity and/or cannabinoid effect comprises activating at least one G protein related enzyme selected from the group consisting of: p38, Raf-1, ERK, JNK, c-fos and c-jun.

4. The method of claim 3 wherein the p38 comprises one selected from the group consisting of: p38α, p38β, p38γ and p38δ.

5. The method of claim 1 wherein the modulated cannabinoid activity and/or cannabinoid effect comprises activating at least one MAPK.

6. The method of claim 5 wherein MAPK activation comprises activating at least one member of a subfamily selected from the group consisting of: ERK1/2, ERK5, JNKs and p38s.

7. The method of claim 1 wherein said at least one cannabinoid related physiological deficit is selected from the group consisting of: poor appetite, overzealous appetite, pain-sensation, lethargy, lack of activity, less than optimal oxytocin, moodiness, memory disorder, emesis, cramps, menstrual pain, spasticity and rheumatism.

8. The method of claim 1 wherein said at least one cannabinoid related physiological deficit involves a disease or condition selected from the group consisting of: neuropathic pain, weight loss, reflex sympathetic dystrophy/causalgia, macular degeneration, peripheral neuropathy, entrapment neuropathy, complex regional pain syndrome, nociceptive pain, neuropathic pain, fibromyalgia, scleroderma, chronic low back pain, visceral pain, general pain, chronic pain, psoriasis, eczema, acute pain, post herpetic neuralgia (PHN), neuropathies, neuralgia, diabetic neuropathy, HIV-related neuropathy, nerve injury, ocular pain, headaches of various etiologies—including migraine, acute herpes zoster (shingles), pain-related disorders such as tactile allodynia and hyperalgesia, rheumatoid arthritic pain, osteoarthritic pain, back pain, cancer pain, dental pain, muscular pain, mastalgia, pain resulting from dermal injuries, fibromyalgia, neuritis, sciatica, inflammation, neurodegenerative disease, cough, broncho-constriction, irritable bowel syndrome (IBS), inflammatory bowel disease (IBD), colitis, emesis, Crohn's disease, ulcerative colitis, asthma, dermatitis, seasonal allergic rhinitis, gastroesophageal reflux disease (GERD), constipation, diarrhea, gastrointestinal disorders, irritable bowel syndrome, cutaneous T cell lymphoma, asthma, dermatitis, multiple sclerosis, osteoarthritis, psoriasis, systemic lupus erythematosus, diabetes, glomerulonephritis, renal ischemia, nephritis, hepatitis, vasculitis, myocardial infarction, cerebral ischemia, chronic obstructive pulmonary disease (COPD), cryptogenic fibrosing alveolitis and bronchitis.

9. The method of claim 1 wherein said at least one cannabinoid related physiological deficit involves a disease related to oxidative stress, said disease being selected from the group consisting of: weight loss, inflammatory pain, cataract, macular degeneration, peripheral neuropathy, visceral pain, acute cerebral ischemia, psoriasis, eczema, stroke, rheumatoid arthritic pain, osteoarthritic pain, osteoarthritis, cerebrovascular ischemia, asthma, dermatitis, psoriasis, osteoporosis, cerebral stroke, vasculitis, myocardial infarction, cerebral ischemia, reversible airway obstruction, adult respiratory disease syndrome, mechanically assisted device induced inflammation and hyperhydrated cornea.

10. The method of claim 1 wherein said combination of cannabinoid activity and/or cannabinoid effect modulating interventions comprises providing at least one chemical or physical intervention selected from the group consisting of: plant cannabinoids, synthetic cannabinoids, processed cannabinoids, exogenously supplied endocannabinoid and exogenously supplied pro-endocannabinoid, to said individual.

11. The method of claim 10 wherein said at least one chemical or physical intervention comprises providing at least one plant cannabinoid selected from the group consisting of:
tetrahydrocannabinol (THC), tetrahydrocannabinolic acid (THCA) and cannabidiol (CBD), products of the cannabis plant); cannabidivarin (CBDV), cannabidiol acid (CBDA), cannabidivarin acid (CBDVA), cannabichromene (CBC), cannabigerol (CBG), cannabigerol acid (CBGA), cannabigerovarin (BGV), cannabinol (CBN), cannabinovarin (CBNV), tetrahydrocannabivarin (THCV), tetrahydrocannabivarin acid (THCVA), delta-8 tetrahydrocannabinol (Δ⁸-THC), pepper, capsaicin and a guineensine inclusive compound not previously listed, to said individual.

12. The method of claim 10 wherein said at least one chemical or physical intervention comprises providing at least one synthetic cannabinoid or processed cannabinoid selected from the group consisting of: HU-210, HU-331, guineensine, JWH015, SATIVEX™ or its generic, dronabinol, nabilone, ajulemic acid, CP 55940, CANNABINOR™ or its generic, methanandamide, THC-11-oic acid and TARANABANT™ or its generic, to said individual.

13. The method of claim 10 wherein said at least one chemical or physical intervention comprises providing at least one exogenously supplied endocannabinoid and exogenously supplied pro-endocannabinoid selected from the group consisting of: AEA, 2AG, PEA, OEA, NADA and O-AEA, to said individual.

14. The method of claim 1 wherein said at least one of said cannabinoid related physiological deficits relates to an endocannabinoid selected from the group consisting of: AEA, 2AG, PEA, OEA, NADA and O-AEA.

15. The method of claim 1 wherein said combination of cannabinoid activity and/or cannabinoid effect modulating interventions comprises providing at least one chemical or physical intervention selected from the group consisting of: a terpene, NO or NO enhancer, sunlight, vitamin D, folate, resveratrol, caffeine, vitamin A, a flavone, genistein, agmatine, kava, TEA, inositol and honokiol, to said individual.

16. The method of claim 15 wherein a flavone selected from the group consisting of: 7-hydroxyflavone and 3,7-dihydroxyflavone is provided to said individual.

17. The method of claim 1 wherein the modulated cannabinoid activity and/or cannabinoid effect comprises modulating activity or expression of a protein selected from the group consisting of: N-arachidonyl-phosphatidylethanolamine (N-arachPE), phospholipase D (PLD), N-acyl phosphatidylethanolamine phospholipase D (NAPE-PLD), glycerophosphodiesterase (GDE1), α, βhydrolase (ABH4), fatty acid amide hydrolase (FAAH), a lysosome-localized fatty acyl amide hydrolase with an acid optimum (NAAA), FAAH-2, PLC, diacylglycerol lipase (DGL), monoacylglycerol lipase (MGL), ABHD6 and ABHD12.

18. The method of claim 1 wherein the modulated cannabinoid activity and/or cannabinoid effect comprises binding at least one selected from the group consisting of: 5-HT_{1A}, 5-HT_{2A}, CB₁, CB₂, TRPV₁, GPR18, GPR55, GPR119, GPR120, PPARα, PPARγ and PPARδ.

19. The method of claim 1 wherein said combination of cannabinoid activity and/or cannabinoid effect modulating interventions comprises delivering a compound selected from the group consisting of: gefitinib, erlotinib, lapatinib, sunitinib, sorafenib, nilotinib, dasatinib, imatinib, tipifarnib, sorafenib, U0126, PD184352, AZD6244, BIX02188/BIX02189, SB203580, SB202190, BIRB-796 and PD98059, to said individual.

20. The method of claim 1 wherein said combination of cannabinoid activity and/or cannabinoid effect modulating interventions selected to ameliorate at least one of said cannabinoid related physiological deficits modules activity of at least one of the following: EGFR, VEGFR, VEGFR, PDGFR, PDGFR, Bcr-Abl, Bcr-Abl/c-Src, Bcr-Abl/c-SCT/ c-Kit/PDGFR, Ras, MEK1/2, MEK1/2, MEK1/2, MEK5, p38α, p38γ, p38δ and MEK1.

21. The method according to claim 1 wherein in said individual having been identified as being compromised by said at least one cannabinoid related physiological deficit, and having been delivered a combination of cannabinoid activity and/or cannabinoid effect modulating interventions selected to ameliorate said at least one of said cannabinoid related physiological deficits with an effect that at least one of said cannabinoid related physiological deficits was ameliorated, at least a second physiological deficit is addressed by delivering to said individual at least one cannabinoid activity and/or cannabinoid effect modulating intervention selected to ameliorate said second cannabinoid related physiological deficit, said at least one physiological deficit selected from the group consisting of a physiological deficit identical in description, but different in magnitude by the ameliorated amount and a physiological deficit having a description differing from said ameliorated deficit.
